(19) United States
(12) Patent Application Publication
(10) Pub. No.: US 2014/0349382 A1
(43) Pub. Date: Nov. 27, 2014

(54) ASSAY OF FUNCTIONAL CELL VIABILITY

(71) Applicant: University of Leeds, Leeds, Yorkshire (GB)

(72) Inventors: Brian Mark Thomson, York (GB); Mark Frederick Smith, York (GB)

(73) Assignee: University of Leeds, Leeds, Yorkshire (GB)

(21) Appl. No.: 14/366,748

(22) PCT Filed: Dec. 21, 2012
(86) PCT No.: PCT/GB2012/053246

§ 371 Date: Jun. 19, 2014

(30) Foreign Application Priority Data
Dec. 21, 2011 (GB) 1121959.9

(51) Int. Cl. G01N 33/50; C12Q 1/02; B01L 3/00

(52) U.S. Cl. CPC G01N 33/5005; B01L 3/5085; C12Q 1/02
USPC 435/288.5; 435/297.5

(57) ABSTRACT

The present invention relates to an apparatus for, and methods of assessing cell viability in a biological sample comprising cells or tissue. In particular the present invention provides quality assurance assays for complex biomaterials, especially but not exclusively cells derived from the pancreas, liver, kidney, lung, bone marrow and stem cells, for use in biomedical procedures. The present invention provides inter alia methods of improving assessment of donor cell/tissue viability, methods of improving the success of donor cell/tissue transplant procedures, methods of assessing the functional properties of complex biological materials prior to their use in regenerative therapies and kits therefor.

The present invention relates to an apparatus for, and methods of, assessing cell viability in a biological sample comprising cells or tissue. In particular the present invention provides quality assurance assays for complex biomaterials, especially but not exclusively cells derived from the pancreas, liver, kidney, lung, bone marrow and stem cells, for use in biomedical procedures. The present invention provides inter alia methods of improving assessment of donor cell/tissue viability, methods of improving the success of donor cell/tissue transplant procedures, methods of assessing the functional properties of complex biological materials prior to their use in regenerative therapies and kits therefor.

### BACKGROUND

Regenerative medicine and transplantation involves treating disease with complex biological materials (e.g. cells, tissues and bioactive extracellular matrices). In contrast, most previous medical techniques treated disease using either simple, chemically-defined, low molecular weight pharmaceutical compounds (e.g. aspirin) or bioinert mechanical implants with tightly defined physical characteristics (e.g. hip replacements).

The complex biological materials used in transplants and regenerative medicine pose a distinct set of quality control concerns for clinicians and regulators because they cannot be statically defined. The assays employed to demonstrate the identity, purity, viability and potency of therapeutics derived from complex biological materials therefore need to (i) assess the test-material's biological functionality instead of its chemical identity or physical properties; and (ii) be performed spatially and temporally close to the point of clinical use.

Therapies requiring improved functional viability assays include, but are not limited to pancreatic islet/organ transplantation, liver transplantation, lung transplantation, kidney transplantation and heart transplantation.

By way of example, pancreatic islet transplantation is a cell-based treatment for type I diabetes complicated by severe hypocalcaemia that is unresponsive to conventional therapy. The procedure has been used internationally since 2000, and is approved and funded by NICE and the NHS Commissioning group for use within the UK.

The quality of islets transplanted into a patient has a marked effect upon clinical outcomes. Whereas transplants of ‘ideal’ islets can lead to 100% five year insulin-independence, only 10% of recipients of ‘less-good’ islets remain insulin independent after 5 years. Transplanting poor quality islets can also exacerbate subsequent immune responses making it more likely that a patient will reject future transplants. Clinicians are therefore reticent to transplant anything except the ‘highest quality’ islets.

To date, there is no internationally recognised quality assurance assay for islet functional viability and ‘islet quality’ can only, at present, be assessed by subjective clinical judgement. This leads to 90% of donated pancreas/isolated islets being discarded unused, despite the limited availability of donor pancreases.

One aspect of the problem is that donated pancreases are often pre-judged following subjective assessment of the organ donor rather than being directly assessed. If quality control assays were available that could identify ‘good quality’ islets from ‘poor quality’ donors it would (i) reduce the waste of donated organs; (ii) improve confidence in the availability of functional islets for transplantation; (iii) and allow clinicians to improve methodology by allowing them to relate observed clinical outcomes to the quality of transplanted islets.

The need for an islet quality control assay is also being driven by the need for clinicians to comply with regulatory statutes (e.g. the Advanced Medicinal products legislation in Europe). For instance, the American FDA have mandated that islet transplantation can only be licensed as ‘standard of care’ in the US if manufacturers are able to demonstrate safety, identity, purity, viability and potency of the transplanted islets. Since existing QC assays cannot deliver compliance with these regulatory systems, a new, widely accepted QC assay for islet viability is urgently required.

By way of a further example, functional viability assays are also required for use with liver transplantation. Advances in liver transplantation have enabled it to become a standard therapy for end-stage liver disease (6200 procedures p.a. in the USA; 600 p.a. in the UK; 68% 5-year survival rate). Unfortunately, the demand for donor organs greatly exceeds supply. This problem could be mitigated by relaxing the criteria used to accept donors (i.e. by using lower quality donated organs), but it is known that the use of marginal donors is associated with increased graft dysfunction and early mortality following liver transplantation.

The underlying problem is that criteria-based donor-assessment systems can only provide broad predictions of expected organ quality (i.e. the best clinical outcomes are generally observed following the use of organs obtained from donors aged between 1-34 years, intermediate results for 35-64 and worst outcomes for donors who were aged <1-year or aged >=65 years) rather than a specific assessment of the functional viability of a particular donated liver immediately prior to its clinical use.

A functional viability assay is therefore required to distinguish between viable and non-viable donor livers that can be performed within the time scales and practical constraints of an operating theatre or clean room. Such an assay could identify viable livers from the marginal donor pool (e.g. donor age >60 years; warm ischaemia time >30 minutes and/or cold ischaemia time >10 hours) and thereby support a clinical decision as to an organ's suitability for use.

An assay that could identify clinically usable livers from amongst those that are currently discarded could increase liver availability by 20-30% (Current Challenges and Controversies in Liver Transplantation; TOUCHBRIEFINGS2007). It might also reduce early graft-failures by identifying the non/less-viable organs that are currently inadvertently used in transplant procedures.

An assay that could rapidly and effectively identify viable donor cells/tissue prior to transplantation would offer immediate benefit to clinicians and patients alike.

An assay that could identify clinically usable cells or tissue, such as stem cells or those derived from the pancreas, liver, lung, kidney, heart, and bone marrow, from those that are currently discarded could increase the availability of cells or tissues for biomedical procedures. It may also reduce early rejection of such cells or tissues by a patient by identifying the non/less-viable cells or tissues that are currently inadvertently used in transplant procedures.

### BRIEF SUMMARY OF THE DISCLOSURE

In one aspect, the invention provides an apparatus for assessing viability of a biological sample comprising cells, the apparatus comprising:

(i) a reagent vessel comprising mutually independent interconnected rows and columns of reagent chambers for containing reagents, each reagent chamber being defined by a front and a rear wall, a pair of side walls and a bottom wall, the reagent vessel comprising a partitioning wall being defined by a side wall of adjacent columns of reagent chambers, the reagent vessel further comprising a means for ensuring correct orientation of an assay cassette within the reagent chambers, wherein the reagent vessel comprises at least one reagent chamber having a cell viability indicator reagent therein, wherein the at least one reagent chamber having a cell viability indicator reagent therein has a removable cover element; and
(ii) an assay cassette comprising at least one reaction chamber housing that encloses at least one reaction chamber, the reaction chamber having an access port for introducing the biological sample therein and a permeable barrier that permits fluid exchange into and out of the reaction chamber whilst retaining the cells of the biological sample within the reaction chamber, the assay cassette further comprising a handling portion.

Optionally, the reagent vessel further comprises at least one reagent chamber having a wash reagent therein, wherein the at least one reagent chamber having a wash reagent therein has a removable cover element.

Optionally, the reagent vessel further comprises at least one reagent chamber having a fixative reagent therein, wherein the at least one reagent chamber having a fixative reagent therein has a removable cover element.

Optionally, the reagent vessel further comprises at least one reagent chamber having a nutrient reagent therein, wherein the at least one reagent chamber having a nutrient reagent therein has a removable cover element.

Optionally, the pair of side walls of each reagent chamber are of commensurate dimensions with one another, the front and rear walls of each reagent chamber are of commensurate dimensions with one another, and wherein the height of the four walls are also commensurate.

Optionally, at least one reagent chamber in each row comprises a means for ensuring correct orientation of the assay cassette within the reagent chambers. Preferably, the means for ensuring correct orientation of the assay cassette within the reagent chambers comprises a profiled region, groove or protrusion located in a side wall of at least one reagent chamber.

Optionally, the removable cover element is a sealable cover, optionally wherein the sealable cover is a tear off cover or lid.

Optionally, the wash reagent comprises PBS.

Optionally, the nutrient reagent comprises glucose, essential amino acids and non-essential amino acids.

Optionally, the nutrient reagent further comprises dithizone or eosin.

Optionally, the cell viability indicator reagent is a tetrazole selected from the group consisting of MTT, XTT, MTS and WSTs. Preferably, the tetrazole is MTT.

Optionally, the cell viability indicator reagent comprises at least one of calcein AM, ethidium bromide HD, propidium iodide, fluorescein diacetate and/or BCECF-AM.

Optionally, the fixative reagent comprises neutral buffered formalin.

Optionally, the reagent vessel further comprises at least one additional reagent chamber having a further wash reagent therein. Preferably, the further wash reagent is PBS or water.

Optionally, the reagent vessel further comprises at least one additional reagent chamber having a devitalising reagent therein. Preferably, the devitalising reagent comprises ethanol.

Optionally, the assay cassette comprises a plurality of reaction chambers, wherein each of the plurality of reaction chambers has a volume commensurate to each of the other reaction chambers.

Optionally, the assay cassette comprises a plurality of reaction chambers, wherein at least two of the plurality of reaction chambers have different shapes.

Optionally, at least a portion of the reaction chamber housing is transparent.

Optionally, the reaction chamber housing is formed, entirely or in part, from a transparent polymer selected from the group comprising polystyrene, polycarbonate or poly methylmethacrylate.

Optionally, the access port of the reaction chamber is either a septal port or is sealed/closed once a biological sample has been placed within the reaction chamber.

Optionally, the assay cassette's external dimensions are approximately length 74 to 77 mm×width 24 to 26 mm×height 1 to 3.5 mm.

Optionally, the reaction chamber housing is formed from a biocompatible or bioinert polymer by injection moulding or by insert-injection moulding.

Optionally, the assay cassette further comprises a cover element for sealing the reaction chambers of assay cassette after use.

Optionally, the assay cassette comprises a base region and the at least one reaction chamber housing depends therefrom. Preferably, the reaction chamber is attached to or formed integrally with the assay cassette base region.

Optionally, the permeable barrier forms a portion of the reaction chamber housing.

Optionally, the permeability of the barrier is either constant across the barrier or varied across the barrier's depth.

Optionally, the permeable barrier is either a two dimensional surface filter or a three dimensional depth filter. Optionally, the permeable barrier is a two dimensional surface filter composed of a woven or non-woven material selected from the group consisting of a polymeric, felt, ceramic or metallic material. Optionally, the permeable barrier is a three dimensional depth filter comprising non-woven fibres or open-celled foams.

Optionally, the assay cassette can only be inserted into the reagent vessel in one of a predetermined number of positions.

Optionally, the handling portion is spaced apart from the reaction chamber housing defining a void region or space therebetween, the void region being sized and shaped so that, in use, either a partitioning wall or a side wall of the reagent vessel can be accommodated therein. Preferably, the assay cassette comprises a plurality of void regions, wherein the number of void regions is proportional to the number of reagent chambers in a row of the reagent vessel.

Optionally, the handling portion of the assay cassette further includes a reference standard, optionally in the form of a multicoloured strip or label.

Optionally, the cells are donor cells.

Optionally, the cells are stem cells, or the cells are derived from the pancreas, liver, lung, kidney, heart or bone marrow.

Accordingly, in one embodiment of the invention, the reagent vessel comprises at least one reagent chamber having a cell viability indicator reagent therein.

In one embodiment, the reagent vessel comprises:
at least one reagent chamber having a wash reagent therein and
at least one reagent chamber having a cell viability indicator reagent therein.

In one embodiment, the reagent vessel comprises:
at least one reagent chamber having wash reagent therein,
at least one reagent chamber having a cell viability indicator reagent therein, and
at least one reagent chamber having a fixative reagent therein.

In one embodiment, the reagent vessel comprises:
at least one reagent chamber having wash reagent therein,
at least one reagent chamber having a nutrient reagent therein
at least one reagent chamber having a cell viability indicator reagent therein, and
at least one reagent chamber having a fixative reagent therein.

In one embodiment, the reagent vessel comprises:
at least one reagent chamber having wash reagent therein,
at least one reagent chamber having a nutrient reagent therein
at least one reagent chamber having a cell viability indicator reagent therein,
at least one reagent chamber having a fixative reagent therein, and
at least one reagent chamber having a further wash reagent therein.

In preferred embodiments of the invention, the reagents within the reagent vessel are provided in discrete reagent chambers. Alternatively, at least two of the reagents within the reagent vessel may be provided in combination in a reagent chamber. By way of example, but not by way of limitation, the wash reagent and nutrient reagents may be provided in combination in the same reagent chamber. In the same manner, any combination of the wash reagent, nutrient reagent, cell viability indicator reagent, fixative reagent, further (or additional) wash reagent, ubiquitous liver indicator reagent and/or intracellular insulin indicator reagent may be provided in combination in the same reagent chamber. The skilled person will readily be able to determine appropriate combinations of such reagents.

In one embodiment, an apparatus for assessing functional and viability donor cells and tissue is provided, the apparatus comprising:

(i) a reagent vessel comprising mutually independent interconnected rows and columns of reagent chambers for containing reagents, the reagent chamber being defined by a front and a rear wall, a pair of side walls and a bottom wall, a partitioning wall being defined by a side wall of adjacent columns of reagent chambers, the reagent vessel further comprising a means for ensuring correct orientation of a test sample within the reagent chambers; and
(ii) an assay cassette comprising a base region and depending therefrom, at least one reaction chamber housing that encloses a reaction chamber, the reaction chamber having an access port for introducing a biological sample therein and a permeable barrier positioned over the reaction chamber main body which permits fluid exchange into and out of the reaction chamber whilst retaining the sample within the reaction chamber main body, the assay cassette further comprising a handling portion that is spaced apart from the said reaction chamber housing defining a void region or space therebetween, the void region being sized and shaped so that, in use, either a partitioning wall or a side wall of the reagent vessel can be accommodated therein.

In a further aspect, the invention provides an in vitro method of assessing the viability of a biological sample comprising cells, the method comprising:
a) introducing the biological sample into at least one reaction chamber of an assay cassette of an apparatus as described herein; and
b) placing the assay cassette into a reagent vessel of the apparatus as described herein,
   wherein the method comprises the step of exposing the biological sample to a cell viability indicator reagent, wherein viable cells are identified by the generation of a first colorimetric signal.

Optionally, the method further comprises the step of exposing the biological sample to a wash reagent.

Optionally, the method further comprises the step of exposing the biological reagent to a fixative reagent.

Optionally, the method further comprises the step of exposing the biological sample to a nutrient reagent.

Optionally, the method further comprises the step of exposing the biological sample to a further wash reagent.

Optionally, the exposing steps are performed in the reagent vessel according to step (b).

Optionally, the wash reagent comprises PBS.

Optionally, the nutrient reagent comprises glucose, essential amino acids and non-essential amino acids.

Optionally, the nutrient reagent further comprises dithizone.

Optionally, the cell viability indicator reagent is a tetrazole selected from the group consisting of MTT, XTT, MTS and WSTs. Preferably, the tetrazole is MTT.

Optionally, the cell viability indicator reagent comprises at least one of calcein AM, ethidium bromide HD, propidium iodide, fluorescein diacetate and/or BCECF-AM.

Optionally, the fixative reagent comprises neutral buffered formalin.

Optionally, the further wash reagent is PBS or water.

Optionally, the cells are donor cells.

Optionally, the cells are stem cells, or the cells are derived from the pancreas, liver, lung, kidney, heart or bone marrow.

In one embodiment, the cells are liver cells and the method further comprises the step of exposing the liver cells to a ubiquitous liver cell indicator reagent, wherein the liver cells are identified by the generation of a second colorimetric signal. Preferably, the ubiquitous liver cell indicator reagent is selected from dithizone or eosin.

In one embodiment, the cells are pancreatic islet cells and the method further comprises maintaining the pancreatic islet cells under conditions for insulin biosynthesis and exposing the pancreatic islet cells to an intracellular insulin indicator reagent, wherein insulin producing pancreatic islet cells are identified by the generation of a second colorimetric signal. Preferably, the pancreatic islet cells are maintained in medium comprising at least one insulin stimulating nutrient.

Optionally, the at least one insulin stimulating nutrient is selected from the group consisting of glucose, alanine and glutamine.

Optionally, the medium is serum free.

Optionally, the intracellular insulin indicator reagent is dithizone.

Optionally, the first colorimetric signal and/or second colorimetric signal is measured using a qualitative, semi-qualitative or fully-quantitative means of analysis.

Optionally, the first colorimetric signal and/or second colorimetric signal is measured by a readily discernible colour change in viable cells.

Optionally, the first colorimetric signal and/or second colorimetric signal is compared to a reference standard or is quantified by an instrument or machine.

Optionally, the first colorimetric signal and/or second colorimetric signal results from a change in luminosity, hue or saturation of pixels in a digital representation or as changes to red, green or blue pixel intensities.

Optionally, the first colorimetric signal and/or second colorimetric signal is assessed using a mathematical function that utilises a plurality of numerical values derived from the red, green or blue pixel intensities comprising a digital image.

Optionally, the first colorimetric signal and/or second colorimetric signal is measured as a change in the ratio between red, green and blue channel intensities for particular pixels within digital images or as log ratio of a plurality of channel intensities contained within a digital image.

Optionally, the method comprises assessing the viability of at least two aliquots of the biological sample, wherein each aliquot is introduced into a distinct reaction chamber of the assay cassette in step (a), and wherein the at least two aliquots are exposed to different conditions such that the cells of one aliquot act as a negative control for cell viability.

Optionally, the negative control is produced by altering the conditions such that the cells of one aliquot are devitalised. Optionally, the cells of one aliquot are exposed to ethanol.

Optionally, the negative control is produced by altering the conditions such that the cells of one aliquot are incubated in a low nutrient containing medium.

Accordingly, in one embodiment of the invention, the method comprises the step of exposing the biological sample to a cell viability indicator reagent.

In one embodiment, the method comprises the steps of:
exposing the biological sample to a wash reagent; and
exposing the biological sample to a cell viability indicator reagent.

In one embodiment, the method comprises the steps of:
exposing the biological sample to a wash reagent;
exposing the biological sample to a cell viability indicator reagent; and
exposing the biological sample to a fixative reagent.

In one embodiment, the method comprises the steps of:
exposing the biological sample to a wash reagent;
exposing the biological sample to a nutrient reagent;
exposing the biological sample to a cell viability indicator reagent; and
exposing the biological sample to a fixative reagent.

In one embodiment, the method comprises the steps of:
exposing the biological sample to a wash reagent;
exposing the biological sample to a nutrient reagent;
exposing the biological sample to a cell viability indicator reagent;
exposing the biological sample to a fixative reagent; and
exposing the biological sample to a further (or additional) wash reagent.

In preferred embodiments of the invention, the method comprises the exposing steps described above in sequential order. In preferred embodiments of the invention, the method comprises the exposing steps in discrete sequential order. Alternatively, the exposing steps of the method are not discrete. By way of example, but not by way of limitation, the biological sample may be exposed to the wash and nutrient reagents simultaneously. In the same manner, the biological sample may be exposed to at least two of the following in any combination: wash reagent, nutrient reagent, cell viability indicator reagent, fixative reagent, further (or additional) wash reagent, ubiquitous liver indicator reagent and/or intracellular insulin indicator reagent. The skilled person will readily be able to determine appropriate combinations of such reagents.

According to a yet a further aspect of the invention there is provided an in vitro method of selecting functional viable donor cells or tissue for regenerative medicine prior to a transplantation procedure, the method comprising:

(i) inserting aliquots of donor cells or tissue into a least one reaction chamber of an assay cassette as hereinbefore described;
(ii) placing the assay cassette into a reagent vessel as hereinbefore described that contains a series of chemical reagents that produce a measurable change or output that is dependent upon the functional viability of the donor cells or tissue; and optionally
(iii) measuring the said change with respect to a control wherein a difference in value from a control is indicative of functional viability.

According to yet a further aspect of the invention there is provided an in vitro method of assessing functional viability of donated organ-derived pancreatic islets prior to an islet transplantation procedure, the method comprising:

(i) inserting aliquots of a pancreatic islet preparation obtained from a donor into two reaction chambers of an assay cassette as hereinbefore described;
(ii) placing the assay cassette into a reagent vessel as hereinbefore described that contains chemical reagents that produce a characteristic colour change when functionally viable beta islet cells are incubated in high nutrient as opposed to low nutrient containing medium; and
(iii) analysing the said colour change by either (a) reference to a colour chart/reference standard or (b) by way of image analysis software.

According to yet a further aspect of the invention there is provided a method of improving success rates of pancreatic islet cell implants into an individual in need of a transplant having type 1 diabetes, the method comprising assessing functional viability of donor organ-derived pancreatic islet according to the method of an aspect of the invention, so as to identify a high quality donated sample prior to implantation.

According to yet a further aspect of the invention there is provided a kit for assessing the viability of a biological sample comprising cells, the kit comprising:

a) an apparatus as hereinbefore described; and
b) instructions for carrying out a method as hereinbefore described.

In one embodiment, the kit further comprises a reference standard. Preferably, the reference standard is for calibrating the visualization of a first colorimetric signal and/or a second colorimetric signal in terms of brightness, orientation, linear dimensions and/or colour balance.

According to a yet further aspect of the invention there is provided a kit of parts, the kit comprising an apparatus according to the first aspect of the invention further including a set of reagents and removable seal or cover for preventing leakage of the reagents and optionally further including a set of instructions and/or a colour reference guide.

According to a yet further aspect of the invention there is provided a kit for determining pancreatic cell functional viability of a donor sample, the kit comprising:

(i) a reagent vessel as hereinbefore described having a removable seal or cover and having a set of reagents comprising (a) high and low nutrient media alone (b) (high and low nutrient media supplemented with) a formazan dye and dithizone solution (c) neutral buffered formalin and (d) phosphate buffered saline;
(ii) an assay cassette as hereinbefore described comprising at least two reaction chambers and optionally further including
(iii) a colour reference guide and/or image analysis software.

### BRIEF DESCRIPTION OF THE DRAWINGS

Embodiments of the invention are further described hereinafter with reference to the accompanying drawings, in which:

FIG. 1a shows a two chambered assay cassette.

FIG. 1b shows a four chambered assay cassette for use with cell suspensions or aggregates (e.g. pancreatic cells).

FIGS. 2a and 2b show a reagent tray in accordance with two embodiments of the invention.

FIG. 3 shows a monochrome representation of live and dead (formalin fixed) ovine pancreatic islets that have been incubated in either low or high nutrient media and then double-labelled with dithizone and MTT.

FIG. 4 shows image analysis of live and dead ovine pancreatic islets incubated in either low or high nutrient media and stained with both dithizone and MTT.

FIG. 5 shows metabolic viability score for islet preparations. The viability of a live (test) islet sample may be compared with a devitalised (negative control) islet sample using MTT and dithizone double labelling and quantitative image processing of light microscopy images.

FIG. 6 shows selected images of ovine islets double labelled with dithizone and MTT. The viability of islets placed in the assay cassette, double-labelled with MTT and dithizone and viewed within the assay cassette by light microscopy may be judged qualitatively and quantified by image processing.

FIG. 7 shows the nutrient concentration required to switch islets from quiescence to metabolic activity.

FIG. 8 shows the minimum time required for islets to respond metabolically to altered nutrient conditions.

FIG. 9 shows the effect of staining upon time log₁₀ (blue/green).

FIG. 10 shows an alternative embodiment of FIG. 1 of an assay cassette for measuring multiple, duplicate aliquots.

FIG. 11 shows an eight chambered assay cassette designed to accommodate needle biopsies (e.g. of solid organs like liver).

FIG. 12 shows a monochrome representation of devitalised and viable human pancreatic islets stained with MTT and dithizone and viewed without a microscope.

FIG. 13 shows a monochrome representation of a calibration image used to standardise the image processing software.

FIG. 14 shows how the large quantity of numerical information generated by the image processing software (e.g. information on the size and metabolic activity of individual islets in a sample) may be summarised and presented to a clinician diagrammatically so as to facilitate rapid appraisal of a sample's quality.

FIG. 15 shows (i) that the staining intensity of MTT and dithizone double labelled cells varies with percentage cell viability within a sample and (ii) that image processing software can readily discriminate between pixels corresponding to stained cells and those corresponding to the background image.

FIG. 16 shows that the results from the image processing-based measure of cell viability (i.e. image analysis of a digital camera photograph of dithizone and MTT double labelled cells performed using software running on a laptop computer) correlates with an established biochemical method for assessing cell viability (i.e. measurement of the absorbance of solubilised formezan dye at 570 nm using a spectrophotometer in a laboratory). Results from image processing test for viability=diamonds (left vertical axis); results from traditional biochemical method for viability using spectrophotometer=squares (right vertical axis).

FIG. 17 shows that the loss of fibroblast cell viability induced by transient hypoxia and re-oxygenation (i.e. a common cause of diminished cell viability in transplant procedures) may be readily identified using the assay.

FIG. 18 shows that the loss of Min-6 cell viability caused by transient hypoxia may be identified in cultures of min-6 pancreatic islet-like cells.

FIG. 19 shows that a scatter graph of fibroblast staining intensity vs stained area distinguishes between human fibroblasts grown under transiently hypoxic instead of normoxic conditions. Normoxia=diamonds; Transient hypoxia=squares.

FIG. 20 shows that a scatter graph of staining intensity vs stained area distinguishes between min-6 pancreatic islet like-cells grown under transiently hypoxic instead of normoxic conditions. Normoxia=diamonds; Transient hypoxia=squares.

FIG. 21 shows a monochrome representation of MTT and dithizone stained fragments of live and devitalised liver tissue.

FIG. 22 shows that the staining intensity of MTT and dithizone stained liver tissue fragments may be assessed by quantitative image processing.

FIG. 23 shows that the MTT and dithizone double labelling method and quantitative image processing routine may be used to monitor the loss of liver tissue viability during a warm ischaemic period of 135 minutes.

FIG. 24 shows that alternative histochemical dyes (e.g. eosin and MTT) may be used to assess liver cell viability in this assay system.

FIG. 25 shows that the image processing routines used in this assay system may be used to quantify the differences in staining patterns of live and dead liver cells stained with eosin and MTT. Dead=diamonds; Live=squares.

FIG. 26 shows a monochrome representation of dead (red; top 3 wells) and live (blue; bottom 3 wells) liver tissue stained with eosin and MTT.

FIG. 27 shows that the image processing routines used in this assay system may be used to quantify the differences in staining patterns of live and dead liver cells stained with eosin and MTT. Live=circles; Dead=squares.

FIG. 28 shows a four chambered assay cassette designed to accommodate needle biopsies (e.g. of solid organs like liver).

FIG. 29 shows the use of a ‘calibration square’ (as in FIG. 13) in the calibration of a digital photograph of an assay cassette in accordance with the invention.

FIG. 30 shows a monochrome image of L929 aggregate stained with MTT using staining method 1. Dark staining (purple in coloured images) denotes viable cells.

FIG. 31 shows the different levels of viability in L929 aggregates cultured under different conditions.

FIG. 32 shows monochrome image of L929 aggregate stained with Calcein AM and Ethidium Bromide Homodimer. Bright staining (green in coloured images) denotes viable cells.

FIG. 33 shows the different levels of viability in kidney, heart and lung samples treated under different conditions.

### DETAILED DESCRIPTION

In accordance with the present invention there is provided methods, apparatuses and kits to assess the viability of a biological sample such as a complex biological material prior to its use in regenerative therapy.

In one aspect, the invention provides an apparatus for assessing viability of a biological sample comprising cells, the apparatus comprising:

(i) a reagent vessel comprising mutually independent interconnected rows and columns of reagent chambers for containing reagents, each reagent chamber being defined by a front and a rear wall, a pair of side walls and a bottom wall, the reagent vessel comprising a partitioning wall being defined by a side wall of adjacent columns of reagent chambers, the reagent vessel further comprising a means for ensuring correct orientation of an assay cassette within the reagent chambers, wherein the reagent vessel comprises at least one reagent chamber having a cell viability indicator reagent therein, wherein the at least one reagent chamber having a cell viability indicator reagent therein has a removable cover element; and
(ii) an assay cassette comprising at least one reaction chamber housing that encloses at least one reaction chamber, the reaction chamber having an access port for introducing the biological sample therein and a permeable barrier that permits fluid exchange into and out of the reaction chamber whilst retaining the cells of the biological sample within the reaction chamber, the assay cassette further comprising a handling portion.

The apparatus can be used in an in vitro method of assessing the viability of a biological sample comprising cells, the method comprising:
a) introducing the biological sample into at least one reaction chamber of an assay cassette of an apparatus as described herein; and
b) placing the assay cassette into a reagent vessel of the apparatus as described herein,
   wherein the method comprises the step of exposing the biological sample to a cell viability indicator reagent, wherein viable cells are identified by the generation of a first colorimetric signal.

Samples of a biological test material may be placed into the assay cassette and immersed in chemicals (e.g. reagents) contained within the reagent vessel or tray to produce a measurable change (e.g. a colorimetric signal) that is dependent upon the viability of the sample. The change (e.g. change in colorimetric signal) may be analysed by qualitative, semi-quantitative or fully-quantitative means. The methods of the present invention provide immediate, qualitative assessment of overall islet viability produce an objective, fully quantitative assessment of islet viability.

The apparatus of the invention may also form part of a kit for assessing the viability of a biological sample comprising cells, the kit comprising:
a) an apparatus as herein described; and
b) instructions for carrying out a method as herein described.

### Assay Cassette

The assay cassette comprises at least one reaction chamber contained within a chamber housing. The chamber housing may be attached to or form part of the assay cassette base portion. The chamber housing encloses at least one reagent chamber (e.g. the chamber housing is shaped such that it forms a reagent chamber).

The assay cassette may contain a plurality of reaction chambers to allow the properties of a complex biological material (e.g. biological sample) to be compared under ‘test’ vs ‘control’ conditions. This arrangement allows the sample's properties to be described in terms of a defining response to an external stimulus.

It will be appreciated that the assay cassette may also comprise a single reaction chamber if the sample under test requires only passage through a series of test reagents.

The plurality of reaction chambers preferably share equal volumes but they may be of differing shapes (e.g. round or square). This arrangement facilitates the identification of which aliquot of a sample was exposed to ‘test’ as opposed to ‘control’ conditions and minimises the risk of operator error when the assay is performed under stressful clinical conditions.

Preferably, each of the reaction chambers are attached to or formed integrally with an assay cassette base portion.

Preferably, each of the reaction chambers is separated from one another by a slit, gap, void region or space defined by outer edges of the reaction chamber housing. The slit, gap, void region or space separating the reaction chambers is sized and shaped so that the dimensions are marginally greater than the width and depth of at least one wall, ideally a partitioning wall and/or a side wall of the reagent vessel, so that in use, once the assay cassette is placed in the reagent vessel the slit, gap, void or space defined by the outer edges of the reaction chamber housing sits over the reagent vessel's reagent chamber wall and the reagent vessel wall is accommodated within the separating portion.

Preferably, the number of separating spaces or void regions is proportional to the number of reagent chambers running parallel in the “x” axis direction.

Preferably, the reaction chamber further includes a permeable barrier, the permeable barrier permits essentially unrestrained fluid exchange into and out of a reaction chamber when placed in the reagent vessel whilst restricting the passage of solid or particulate biological test materials into or out of the chamber and into a reagent chamber. This arrangement allows a biological sample contained within a reaction chamber to be exposed to a sequence of reagents without the sample being lost from the assay cassette.

Preferably, the permeable barrier forms a portion of the reaction chamber housing (e.g. the barrier may represent one of the “walls” of the reaction chamber housing that encloses or partially encloses the reaction chamber).

Preferably, the reaction chamber has opposing front and rear walls. In some embodiments of the invention, for example when assaying pancreatic cells, the reaction chamber is provided with at least one solid wall and the opposing wall is provided by the permeable barrier. In other embodiments where the biological sample being assayed is a tissue or a cellular aggregation, the opposing front and rear walls may each be provided by the permeable barriers. It will be appreciated that the reaction chamber acts to encapsulate the biological sample being assayed whilst simultaneously allowing reagents access to the biological sample through the at least one permeable barrier.

In some embodiments of the invention, the permeability or porosity of the permeable barrier may be essentially constant across the barrier or may vary across the barrier's depth.

Preferably, the permeable barrier comprises an essentially 2 dimensional surface filter (e.g. a sieve or screen). Alternatively the permeable barrier may comprise a 3 dimensional depth filter (e.g. an open-celled foam).

In the instance where the invention is used to assay the viability of large particulates (e.g. cell aggregates, for instance pancreatic islets; or tissue biopsies, for instance liver biopsies) then the permeable barrier may comprise an essentially two dimensional surface filter. Preferably, the two dimensional structure may be composed of a material selected from the group comprising a polymeric, ceramic, felt, or metallic material. In a preferred embodiment the permeable barrier consists of a nylon mesh. In a more preferred embodiment the permeable barrier consists of a nylon mesh of pore size 40-80 μm. More preferably, the permeable barrier consists of a nylon mesh of pore size of 40, 45, 50, 55, 60, 65, 70, 75, or 80 μm (or any range thereinbetween). In the instance where the present invention is used for assaying single cells (including bone marrow samples) the permeable barrier may comprises a depth filter of varying effective pore size (0.1 μm-200 μm).

In alternative embodiments the two dimensional structure may be composed of any woven or non-woven construction in order to achieve the desired filtration properties (e.g. by achieving a required combination of pore size and open-area). Examples include non-woven felts, plain-weaves or more specialised planar structures (e.g. Hollander Twill weaves).

Where the permeable barrier consists of a three dimensional depth filter, then it may be composed of any of a wide range of structures known to those skilled in the art. Examples include bonded non-woven fibres and open-celled foams.

Preferably, the permeable barrier may be bonded onto the reaction chamber housing (e.g. onto the body of the reaction chamber housing) by heating (e.g. by ultrasonic welding, heated dies, radio frequency sealing). Alternatively, it may be attached to the chamber housing by adhesives. Alternatively it may be attached to the chamber housing physically by way of clips, staples, pins or the like. In certain embodiments the permeable barrier may be incorporated into the (body of the) reaction chamber housing (e.g. via insert injection moulding).

In certain embodiments a portion of the assay cassette is transparent in order to permit observation of the reaction chambers' contents. In a preferred embodiment the reaction chamber includes a transparent portion that permits the contents of the reaction chamber to be observed directly by microscopy. In certain embodiments the transparent portion of the assay cassette consists of thin sheets of plastic or glass (e.g. coverslips). The transparent portion of the reaction chamber may be bonded to the rest of the reaction chamber by heating (e.g. by ultrasonic welding, heated dies, radio frequency sealing or insert moulding).

In alternative embodiments the assay cassette's enclosure may be formed, entirely or in part, from a transparent polymer. Examples include polystyrene, polycarbonate or poly methylmethacrylate. This arrangement facilitates both manufacture and the observation of the contents of the chambers in the assay cassette.

The biological test materials may be introduced into the reaction chambers via apertures or access ports in the periphery of the reaction chamber e.g. at the area adjacent the assay cassette base portion. These apertures may be sealed/closed once the biological samples are in place within the reaction chamber. Alternatively, the biological test material may be injected into the chambers though a septal port. Septal ports may be composed of materials known to those skilled in the art, (e.g. butyl rubber).

The assay cassette of the present invention may further include a reference guide (also termed a reference standard herein). Ideally this may be positioned adjacent the reaction chambers and separated therefrom by a slit, void region or gap that in use accommodates a wall of the reagent chamber of the reagent vessel. Preferably the reference standard may be in the form of a multicoloured strip or label. The multicoloured strip may act as a guide to qualitative assessment of the assay results or as a reference standard for use during quantitative image analysis. Example colours include (i) white (to enable an image processing programme to assess the colour balance and brightness of the illumination that has been used to observe the specimen) and (ii) the colour of a dye produced by an assay reaction (e.g. Pantone 7428 Dark crimson).

Preferably, the assay cassette's external dimensions may be selected to facilitate its examination using a standard optical microscope and stage. In a preferred embodiment the assay cassette is of similar external dimensions to a standard microscope slide (i.e. the assay cassette's external dimensions are approximately length 74 to 77 mm×width 24 to 26 mm×height 1 to 3.5 mm, preferably, length 76 mm×width 25 mm×height 1-3.5 mm). This allows the assay cassette and the biological sample it contains to be examined in more detail (e.g. by microscopy) if required.

Advantageously, the external dimensions and shape of the assay cassette may allow it to be inserted into a reagent vessel (as described hereinafter) in one of a restricted number (e.g. a predetermined number) of possible orientations or positions.

Preferably, the reaction chamber housing may be formed from a biocompatible or bioinert polymer. In certain embodiments the reaction chamber housing is formed from a thermoplastic by injection moulding or by insert-injection moulding.

Preferably, the assay cassette is manufactured under clean conditions and/or sterilised prior to distribution or use. In certain embodiments the assay cassettes may be sterilised by methods known to those skilled in the art (e.g. gamma irradiation or ethylene oxide treatment).

In certain embodiments the assay cassette of the present invention may include an additional component to cover or seal the assay cassette after use. In particular, this component may cover the permeable barriers and/or the apertures or ports through which the samples are inserted, so as to prevent liquid from leaving the reaction chambers whilst the samples in the reaction chambers are being observed. This covering component may be separate from the assay cassette or may be a closable lid that forms part of the assay cassette (e.g. a clam-shell like arrangement). Alternatively the cover may consist of an impermeable sheet that may be bound to the enclosure (e.g. with an adhesive).

The present invention relates to apparatuses and methods that may be used for assessing viability of biological samples comprising cells. The skilled person will appreciate that the present invention is intended to allow the identification of cells that may be of use in transplantation, and that in this context, it may be preferred to identify cells that are not only viable, but also able to exert their biological functions required for their therapeutic use. Thus, the present invention provides methods and apparatuses that can be used not only to assess viability of cells, but also functional viability, in which biological status or function of the cells in question is also assessed, normally by means of a further suitable reagent (for example the ubiquitous liver cell indicator reagent or intracellular insulin indicator reagents described below).

It will be appreciated that, since they include an assessment of viability, all aspects or embodiments of the invention that are described as suitable for assessing functional viability of biological samples comprising cells will also be suitable for assessing the viability alone of such samples.

For the purposes of the present invention, a “biological sample comprising cells” may be any biological sample of interest in which cells are present. Merely by way of example, a suitable sample may comprise isolated biological cells. Suitably, a biological sample comprising cells may be a somewhat more complex sample. More complex samples may include tissue or organ samples comprising cells. Examples of such samples include samples derived from the lungs, liver, or other sources considered in further detail elsewhere in the specification. A biological sample comprising cells may be a test material in which it is wished to use the methods or apparatuses of the invention to assess viability or functional viability with a view to possible therapeutic use of the material (such as a donor organ) from which the test material is derived.

By way of example, the biological sample may comprise beta cells. As used herein, “beta cells” refer to pancreatic islet cells which produce, store and secrete insulin.

The biological sample may be an isolated pancreatic islet cell preparation. As used herein, “isolated pancreatic islet cell preparation”, “isolated islets” or “islet sample” all refer to a preparation comprising islet cells isolated from a donor pancreas, including beta cells. Preferably, the islet cells isolated from a donor pancreas are suspended in a physiological saline buffer, (e.g. phosphate buffered saline), a culture medium (e.g. DMEM) or a specialised solution designed to maximise the retention of organ viability prior to transplantation (e.g. University of Wisconsin solution). Alternatively, the isolated pancreatic islet cells preparation may be a pancreatic tissue biopsy.

### Reagent Vessel

The reagent vessel is a container or tray for accommodating/containing and compartmentalising the reagents used in the viability assay. The reagent vessel or tray consists of a multiwell plate comprising an array of reagent chambers for holding reagents. Reference herein to a “reagent chamber” is intended to include a cavity or enclosure or well for holding the reagent, the terms are interchangeable.

The reagent chambers comprise a pair of side walls, front and rear walls, and a bottom wall. Preferably, the side walls are of commensurate dimensions with one another. Preferably the front and rear walls are of commensurate dimensions with one another. Preferably, the height of the four walls are commensurate although the length of the two pairs of walls may be different. The side, front and rear walls are arranged to be at right angles with respect to one and are joined at their bases to the bottom wall/floor. The reagent chamber is open at an end opposite to the floor/bottom wall. A typical reagent chamber approximates to an open box.

In one embodiment, the reagent vessel comprises at least one reagent chamber having a wash reagent therein. As used herein, the term “wash reagent” refers to a reagent that reduces or substantially removes from the biological sample any undefined contaminants or components that may affect the assay (or the assessment of the results of the assay) of the invention.

By way of example, the wash reagent may substantially remove the (undefined) transport medium that previously held the cell sample e.g. islet sample. The isolated islets arrive in a transport medium (e.g. University of Wisconsin Solution), but the composition of that solution will be unknown and cannot be specified in advance. Furthermore, the nutrients in the transport medium will have been consumed to an unpredictable extent whilst adverse materials may have accumulated to potentially toxic levels (e.g. inflammatory cytokines, cell debris and metabolic waste products).

Ideally, the wash solution may be sterile, chemically stable (e.g. have a shelf-life of more than 6 months at 4° C.), isotonic and/or biocompatible (e.g. composed of cell culture grade non-cytotoxic materials).

Preferably it should be buffered, for example to maintain a pH of between 6.4 and 7.8 at room temperature in a normal air atmosphere (e.g. without the need for a humidified atmosphere containing 5% CO₂). Ideally it does not contain any of the strongly coloured components that are sometimes added to culture media (e.g. phenol red).

In a preferred embodiment, the wash solution comprises PBS. Results show that Dulbecco's phosphate buffered saline (Sigma D8537) may be used. However, it is expected that other neutral phosphate buffered saline solutions could be used instead without adversely influencing the results.

Preferably, in the context of the method of the invention, a biological sample is exposed to a wash reagent such that the wash reagent reduces or substantially removes undefined contaminants or components that may affect the assay of the invention from the biological sample. Suitable exposure times can easily be determined by the person skilled in the art. However, a suitable range of exposure times may be from 1 to 15 minutes, preferably from 3 to 7 minutes, more preferably approximately 5 minutes. An exposure time of any one of 1, 2, 3, 4, 5, 6, 7, 8, 9, 10, 11, 12, 13, 14, or 15 minutes (or any range thereinbetween) may also be suitable.

In one embodiment, the reagent vessel comprises at least one reagent chamber having a devitalising reagent therein. As used herein, the term “devitalising reagent” refers to a reagent that devitalises the cell sample i.e. permanently reduces the metabolic activity of the cells in the biological sample to basal levels. Preferably, the devitalising reagent kills the cells in the biological sample.

Preferably, the devitalising reagent is used to produce a ‘negative control’ sample against which a test sample of unknown viability may be compared. Ideally, the devitalising reagent rapidly kills the cells in the negative control sample without dramatically altering cell morphology. Preferably, the devitalising reagent is not so toxic that it creates a hazard to health during the assay's use or subsequent waste disposal (e.g. azide may not be desirable).

Preferably, the devitalising reagent comprises ethanol. A range of ethanol concentrations may be used. Suitable ethanol concentrations can easily be determined by the person skilled in the art. Suitable ethanol concentration ranges may include 50-100%, preferably 70-100%, more preferably 70-95%. In a preferred embodiment, the devitalising reagent comprises 70% ethanol.

Preferably, the devitalising reagent comprises wash reagent. For example, in a preferred embodiment, the devitalising reagent is 70% ethanol in PBS.

Preferably, in the context of the method of the invention, a biological sample (negative control) is exposed to a devitalising reagent such that the devitalising reagent permanently reduces the metabolic activity of the cells in the sample to basal levels. Suitable exposure times can easily be determined by the person skilled in the art. However, a suitable range of exposure times may be from 1 to 15 minutes, preferably from 3 to 7 minutes, more preferably approximately 5 minutes. An exposure time of any one of 1, 2, 3, 4, 5, 6, 7, 8, 9, 10, 11, 12, 13, 14, or 15 minutes (or any range thereinbetween) may also be suitable.

A 5 minute incubation period typically used herein therefore provides a ‘safety margin’ to mitigate against user variability, impatience or error. Longer time periods (e.g. 7 minutes or 15 minutes) are functional and do not have any adverse consequences beyond introducing unnecessary delay.

In one embodiment, the reagent vessel comprises at least one reagent chamber having a nutrient reagent therein. As used herein, the term “nutrient reagent” refers to nutrient reagents comprising “high” nutrient concentrations and nutrient reagents comprising “low” nutrient concentrations.

Preferably, the nutrient reagent comprises a high nutrient reagent that maintains or restores metabolic activity in viable cells in the biological sample.

After post-mortem recovery of cells e.g. pancreatic cells from an organ donor, enzymatic isolation of the islets from the donated pancreas and (potentially) prolonged exposure to sub-optimal cell culture conditions (e.g. transport between hospitals in University of Wisconsin Solution) a biological sample is likely to be traumatised and temporarily metabolically dormant, irrespective of its actual viability.

A high nutrient reagent is used within the context of the method of the invention to allow viable cells in the biological sample to recover and achieve metabolic activity. Preferably, the nutrient reagent comprises glucose and non-essential amino acids.

The concentration range of the non-essential amino acids in the high nutrient reagent may vary. Preferably, the non-essential amino acids are used at manufacturers recommended concentration as a 1 in 100 v/v dilution of commercially available stock (i.e. 1%). However, a concentration from 0.5% to 5% may also be used.

The glucose concentration in the high nutrient reagent may also vary. It is desirable to maximally stimulate insulin production (via increased Beta islet cell metabolism) so a concentration range of 17 mM-25 mM glucose is preferred (although higher concentrations may also be used (e.g. up to 50 mM).

Preferably, in the context of the method of the invention, a biological sample is exposed to a high nutrient reagent such that the high nutrient reagent maintains or restores metabolic activity in viable cells in the biological sample. Suitable exposure times can easily be determined by the person skilled in the art. However, a suitable range of exposure times may be from 15 to 90 minutes, preferably from 30 to 90 minutes, more preferably from 30 to 45 minutes, most preferably from 40 to 45 minutes. Any range thereinbetween may also be suitable.

Results also showed, surprisingly, that although islets respond to changes in nutrient levels very rapidly in vivo, (i.e. <=15 minutes) much longer time periods of exposure to high nutrients are required to restore full metabolic activity to traumatised/quiescent isolated islet samples in vitro. Preferred time periods for incubation in the (high) nutrient reagent are therefore more than 30 minutes, ideally 40-45 minutes. Longer time periods (60-90 minutes) are likely to have beneficial effects in scientific studies because they will provide a more stable baseline, but are likely to introduce unnecessary delays when the assays are used in a clinical situation

Results from studies using human, sheep and rat islets indicate that exposure to high nutrient PBS (i.e. PBS supplemented with 25 mM glucose plus non-essential amino acids) for approximately 40 minutes constitutes a simple, chemically-defined mechanism for inducing high metabolic activity in previously quiescent isolated islets. This solution will also maintain the pH without the need for a CO₂ buffered incubator.

Additional components may be added to this nutrient reagent to provide a more complete nutrient mixture with a broader range of stimuli, (e.g. by adding 10% Ham's F-12). Other components that may be added include essential amino acids and/or synthetic chemicals known to stimulate islet cell metabolism e.g. succinate methyl ester.

The reagent vessel comprises at least one reagent chamber having a cell viability indicator reagent therein. As used herein, the term “cell viability indicator reagent” refers to a reagent that produces a colorimetric signal after exposure to viable cells such that it can be used to identify viable cells in a biological sample.

The cell viability indicator can be any substance, composition or compound capable of providing a colour change which selectively identifies the presence of viable cells in the biological sample. Preferably, the cell viability indicator is a tetrazole, more preferably a tetrazole selected from 3-(4,5-Dimethylthiazol-2-yl)-2,5-diphenyltetrazolium bromide (MTT), 2,3-bis-(2-methoxy-4-nitro-5-sulfophenyl)-2H-tetrazolium-5-carboxanilide (XTT), 3-(4,5-dimethylthiazol-2-yl)-5-(3-carboxymethoxyphenyl)-2-(4-sulfophenyl)-2H-tetrazolium (MTS) or Water soluble Tetrazolium salts (WTS's), for example WST-1 and WST-8 (2-(2-methoxy-4-nitrophenyl)-3-(4-nitrophenyl)-5-(2,4-disulfophenyl)-2H-tetrazolium). Tetrazoles serve as a substrate for an enzymatic reaction, which provides a colorimetric measure of the activity of cellular metabolic enzymes that reduce the tetrazoles to formazan. In a preferred embodiment the cell viability indicator is MTT, and the first colorimetric signal is the generation of a purple coloured formazan upon reduction of MTT by metabolically active cells.

Viable, metabolically active islets are stained blue by MTT. By comparison, the devitalised islets in the negative control sample are unstained or stained faintly brown-yellow. Because islets stained with MTT are visible to the naked eye (as small dark dots) the use of this dye allows a clinician to gain an initial, qualitative result before the assay is complete and quantification has provided a detailed numerical output.

A range of MTT concentrations may be suitable and can easily be determined by a skilled person in the art. By way of example, MTT concentrations of 1 to 2 mg/ml may be used. A concentration of 2 mg/ml is most preferred as it provides an excess of reagent and therefore is less likely to be adversely affected by small losses of reagents during assay storage.

Preferably, in the context of the method of the invention, a biological sample is exposed to a cell viability indicator reagent such that the cell viability indicator reagent produces a colorimetric signal that it can be used to identify viable cells in a biological sample. Suitable exposure times can easily be determined by the person skilled in the art. However, a suitable range of exposure times may be from 5 to 20 minutes, preferably from 10 to 20, more preferably 15 to 20 minutes, and most preferably approximately 15 minutes. Any range thereinbetween may also be suitable.

The inventors have observed that there was an initial ‘lag’ period of ˜5 minutes before MTT staining became visible and it was therefore concluded that incubation periods of 10 minutes or less would be vulnerable to increased variability if the incubation time was incorrectly applied. By contrast, the staining produced by an incubation time of 15 minutes would less vulnerable to small errors by the assay's users.

Other suitable cell viability indicator reagents may also be used.

In certain embodiments of the invention biological samples are exposed to fluorescent dyes to provide information regarding the biological function of the cells within the sample. Such fluorescent dyes include ‘live cell’ dyes (e.g. calcein AM) which selectively accumulate within viable cells and which are modified within the environment of viable cells to produce fluorescent chemical species. Such ‘live cell’ dyes selectively render viable cells fluorescent whilst leaving non-viable cells unstained. Variants of these ‘live cell’ dyes have chemical groups such that they become covalently attached to cellular proteins during fixation so that the dye is retained within the cell for prolonged periods of time.

Other fluorescent dyes include ‘dead cell’ dyes (e.g. propidium iodide or ethidium bromide homodimer) which can enter and stain non-viable cells but which are excluded from viable cells.

In one embodiment, the reagent vessel comprises at least one reagent chamber having a fixative reagent therein. As used herein, the term “fixative reagent” refers to a reagent that terminates the staining reaction and preferably kills any pathogens present in the biological sample.

Preferably, the fixative reagent comprises neutral buffered formalin. However, any other suitable fixative reagents may also be used.

Typically, 10% NBF is used (as supplied by the manufacturer. This is 3.7%-4.0% formaldehyde w/v in PBS). However, a range of concentrations may be used, from e.g. 1% to 10%.

Preferably, in the context of the method of the invention, a biological sample is exposed to a fixative reagent such that the fixative reagent terminates the staining reaction and preferably kills any pathogens present in the biological sample. Suitable exposure times can easily be determined by the person skilled in the art. However, a suitable range of exposure times may be from 15 seconds to 30 minutes, preferably 1 minute to 15 minutes, more preferably 5 minutes to 15 minutes.

In one embodiment, the reagent vessel comprises at least one reagent chamber having an additional wash reagent therein. Preferably, the additional wash reagent is PBS or water.

Preferably, in the context of the method of the invention, a biological sample is exposed to an additional wash reagent to reduce or substantially remove from the biological sample any undefined contaminants or components that may affect the assessment of the results of the assay. Suitable exposure times can easily be determined by the person skilled in the art. However, a suitable range of exposure times may be from 1 to 45 minutes, preferably 1 to 5 minutes.

In certain embodiments the invention biological samples are exposed to additional reagents that provide further information regarding the biological function of the cells within the sample. Such reagents include histological stains, the use of which may improve the sensitivity and discriminating power of the assay.

One example of an additional reagent of this type is an intracellular insulin indicator reagent. A suitable intracellular insulin indicator reagent is any reagent that is able to distinguish those cells in which insulin is present from those in which insulin is largely absent.

The histological stain dithizone is an example of a suitable intracellular insulin indicator reagent which may be employed in the methods or apparatuses of the invention. When islets produce insulin it is stored as a zinc complex prior to its release. The pink coloured zinc chelating reagent dithizone therefore stains insulin-rich islets pink. In contrast, since dead/dying islets degranulate and release their insulin in an uncontrolled manner, dysfunctional islets show much lower levels of dithizone staining.

Dithizone staining is a comparatively slow process and including it as a separate step in the method of the invention may delay the results from the assay. It can however be added to the nutrient reagent to give an additional marker of islet functional viability without creating additional delay. Thus in a preferred embodiment, when an intracellular insulin indicator reagent is used it may be provided in the nutrient reagent.

Alternatively, the intracellular insulin indicator reagent may be a detectably labelled anti-insulin antibody, such as a guinea pig anti-human insulin; Abcam ab7842, which can be detected using immunocytochemical methods.

In embodiments employing intracellular insulin indicator reagents it may also be desired to stimulate insulin production, in order to increase the level of insulin staining that may be achieved. In embodiments of this sort it may be wished to expose the cells of the biological sample to an insulin stimulating nutrient. The skilled person will be aware of many such suitable nutrients, including glucose, and/or synthetic chemicals e.g. succinate esters.

In one embodiment of the method of the invention comprises maintaining pancreatic islet cells under conditions for insulin biosynthesis. As used herein, “maintaining pancreatic islet cells under conditions for insulin biosynthesis”, refers to providing a biological sample comprising pancreatic islet cells with the necessary physiological conditions to biosynthesize insulin, for example conditions that permit or promote insulin biosynthesis. Preferably, the biological sample is provided with a high nutrient medium, comprising at least one agent known to promote insulin production, for example comprising at least one of glucose, alanine or glutamine. Preferably, the medium is serum free. A suitable high nutrient medium comprises phosphate buffered saline supplemented with glucose (25 mM), non-essential amino acids (Sigma M7145; i.e. alanine 8.9 mg/l; asparagine 15 mg/l; aspartate 13.3 mg/ml; glutamate 14.7 mg/l; glycine 7.5 mg/l; proline 11.5 mg/l; and serine 11.5 mg/l) and glutamine (2 mM).

A further example of an additional reagent that may be used in the methods or apparatuses of the invention is a ubiquitous liver cell indicator reagent. A suitable ubiquitous liver cell indicator reagent is any reagent that is able to stain or otherwise label liver cells within a sample.

Dithizone represents a suitable example of a ubiquitous liver cell indicator reagent, by virtue of its ability to dye liver cells that contain zinc. Other histological stains such as eosin may also be used as examples of ubiquitous liver cell indicator reagents.

A combination of ‘live cell’ and ‘dead cell’ fluorescent dyes may be used to reveal the viability of a biological sample.

The reagents may be liquids, solutions, emulsions or gels. In particular embodiments the reagents may comprise materials selected from a list, including but not limited to, culture media, nutrients, hormones, growth factors, pharmaceuticals, biomaterials, extracellular matrix components, dyes, histochemical stains, chromophores, fluorochromes, fixatives, buffers, saline, water and mixtures thereof.

The reagents may be arranged in the reagent vessel or tray to facilitate the assessment of the test material's biological status or function. In certain embodiments the reagents may be arranged in the reagent tray to enable the assessment of the test material's biological status or function, such as viability, by sequential exposure of the test material to a plurality of reagents.

In certain embodiments of the invention, exposure of the test material to the reagents contained within the reagent tray leads to the production of a measurable change that is dependent upon the test material's biological status or function.

In certain embodiments sequential exposure of the test materials contained within the assay cassette to the reagents contained within the reagent tray results in a measurable change (e.g. a colorimetric signal) in the optical properties of the materials within the assay cassette and/or reagent tray thereby permitting the biological status or functionality of the test material to be determined.

The reagent chambers within the reagent tray are mutually independent or discrete and may be disposed as an orthogonal array. In particular embodiments the spacing and arrangement of the cavities permits the positioning of the assay cassette so that the reaction chambers are immersed into the reagents contained within the cavities of the reagent tray.

In particular embodiments the reagent chambers are arranged preferably in contiguous, parallel columns or rows so as to facilitate the assessment of aliquots of a test material under ‘treated’ and ‘control’ conditions. It will be appreciated that the number of reagent chambers is dependent on the number of reagents, for example if an assay employs six different reagents then the reagent vessel or tray will comprise six discrete mutually independent reagent chambers in a “x” axis direction (rows). Furthermore, if a user wishes to run several test samples contemporaneously, for example four, then the reagent vessel will be in the form of a six (“x” axis; rows) by four (“y” axis; columns) grid or multi-well plate.

In certain embodiments the spacing, arrangement, shape, size or wall thickness of the cavities restricts the range of possible orientations in which the assay cassette may be inserted into the reagent tray. This restriction may be utilised to reduce the possibility of operator error. For example, in the instance where the assay cassette comprises a reference standard portion, the assay cassette and reagent vessel are designed so that it is not possible for a user to insert the reference standard portion into the reaction vessel. Alternatively, in instances where the assay cassette does not comprise a reference standard portion, the central partitioning wall between rows/columns of reagent chambers and/or side walls of the reagent vessel may be sized so that it is not possible to insert the assay cassette into the reagent vessel in any other orientation than the correct one.

Preferably, the reagent vessel of the present invention includes a series of profiled regions, protrusions or grooves, the number of which is commensurate with the number of reagent chambers in the “x” axis direction (rows).

Preferably, the profiled regions, protrusions or grooves are sized and shaped to restrict movement of the assay cassette when inserted into the reagent vessel. Movement is ideally restricted so that the assay cassette cannot fall over, tip or be totally submerged in the reagent vessel but allows a minimal amount of movement to agitate the reagent if required.

The reagent vessel may either be supplied without the reagents or pre-loaded with reagents (e.g. by a manufacturer). If the reagent tray is supplied pre-loaded with reagents then the reagents may be retained in the reagent chambers by a removable cover element. For example, the reagent vessel may be fitted with a ‘tear-off’ cover or lid to prevent spillage or contamination before use (i.e. during storage or transit).

Preferably, the reagent tray when supplied as a kit, is supplied with a ‘tear-off’ cover to prevent spillage or contamination during storage or transit. In certain embodiments the ‘tear-off’ cover may incorporate tear tapes or ribbons. This ‘tear-off’ cover is removed immediately before the assay is used. In certain embodiments the ‘tear-off’ cover or lid may be bonded to the reagent tray with an adhesive. Alternatively the ‘tear-off’ cover or lid may be bonded to the reagent tray using heat (e.g. by ultrasonic welding, heated dies or radio frequency sealing). Alternatively physical methods may be used to attach the cover to the reagent tray (e.g. fold over lips).

In certain embodiments the reagent tray may include labels or stickers to supply or record information (e.g. instructions for use).

### Means of Analysis

The assay's end-point may be assessed by qualitative, semi-quantitative or fully-quantitative means of analysis.

In the instance where the output from the assay is judged qualitatively, results may be appraised by direct observation of the assay cassette and/or reagent vessel. Examples include instances where the assay produces a readily discernable colour change in the reagents or test materials dependent upon the status or functional viability of the test material.

In the instance where the output from the invention is to be judged semi-quantitatively, the output from the assay may be compared to a colour chart or reference standards. These colour charts or reference standards may be present on the assay cassette itself, reagent vessel, accompanying literature or the packaging of the kit.

Where the result from the invention is to be judged fully-quantitatively the output may be quantified by an instrument or machine.

In certain embodiments a colour change generated by the invention may be measured directly (e.g. by a spectrophotometer). Alternatively, the colour change may be recorded in an intermediate form prior to subsequent analysis (e.g. by photography or digital imaging of the assay cassette followed by computerised image analysis).

In embodiments where the data are to be fully-quantified and where the output from the invention constitutes an optical change (e.g. a colour change in the reagent or test material) then the results may be assessed by quantitative image analysis.

In certain embodiments the output from the invention may be summarised as changes in the luminosity, hue or saturation of the pixels in a digital representation. Alternatively the output may be summarised as changes to the red, green or blue pixel intensities. The raw data from the digital representation may be processed using techniques known to those skilled in the art of image analysis (e.g. thresholding or local/adaptive thresholding).

In a preferred embodiment the output of the invention may be summarised using a mathematical function that utilises a plurality of numerical values derived from the red, green or blue pixel intensities comprising a digital image. Examples include (i) changes in the ratio between the red, green and blue channel intensities for particular pixels within digital images and (ii) comparison of pixel values with background values. In a most preferred embodiment, the output from the invention is summarised using the log ratio of a plurality of channel intensities contained within a digital image. These log ratios may be used to enumerate histological staining. In particular, they may be used to enumerate differences in histological staining between samples of complex biological materials cultured under ‘test’ as opposed to ‘control’ conditions.

In a most preferred embodiment the result of the assay is quantified by placing the stained assay cassette containing the biological samples on the appropriate region of the reagent tray, photographing the stained assay cassette with a standard digital camera (e.g. a Panasonic Lumix DMC FZ45) and then analysing the digital images using custom software running on computer. This arrangement allows the sample to be quantified close to the point of use and avoids the delays associated with transporting samples to a laboratory.

In a preferred embodiment the numerical data generated by the software is summarised diagrammatically to facilitate clinical interpretation. In a further preferred embodiment the numerical data generated (e.g. the number, size and staining intensities of islets within a sample) may be exported to a spread sheet for further analysis.

In a preferred embodiment the assay cassette comprises a reference standard (e.g. a multicoloured strip). The multicoloured strip (used to calibrate the image analysis of the assay cassette) may comprise a rectangular block of 256 stripes of grey that range in intensity from white to black. The graduated grey block has a pre-determined size (e.g. 20 mm×25 mm) and a predetermined position on the assay cassette (e.g. the centre of the calibration strip is 30.55, 40.45, 56.55 and 66.45 mm to the left of the centres of the four reaction chambers).

This graduated grey block is bordered by a red line adjacent to the white edge, a blue line along the top edge of the graduated block and a green line along the bottom of the graduated block.

Image analysis software may identify various features of this multicoloured calibration block in photographs of the stained assay cassette and then use this information to calibrate the staining of biological samples held within the reaction chambers of the assay cassette.

For instance, the number of pixels between the mid points of the red line and black edge of the reference block (i.e. the left and right margins of the reference block) correspond to the number of pixels in 20 mm. Likewise, the midline of the assay cassette maybe calculated (e.g. by linear regression) from sets of points identified on the blue and green lines. Taken together with the known geometry of the assay cassette and known geometric equations (e.g. the equation of an ellipse), the information gathered from the reference block allows photographs of the assays cassette to be mapped by automatic image analysis systems, without prior knowledge of the scale of the photograph, the light conditions under which the photograph was taken, or the orientation of the cassette within a photograph.

Likewise, comparison of (i) the ‘measured’ red, green and blue channel intensities of a pixel at a given location within a photograph of the calibration strip, with (ii) the ‘true’ rgb values of that position within the calibration strip, allows a ‘look-up-table’ (LUT) to be constructed that relates ‘measured’ to ‘true’ pixel intensities. For instance, the mid-point of the calibration strip should have pixel intensities of red=128, green=128 and blue=128. Any discrepancy between those ‘true’ values and the ‘measured’ values observed on a photograph of the calibration strip (e.g. due to incorrect exposure or poorly white-balanced lighting conditions) may be corrected for using arithmetical procedures known to those skilled in the art. Any ‘gaps’ in the resulting LUT may be filled by interpolation.

Once the size, orientation and location of the assay cassette has been determined within a photograph, (and the position of the chambers calculated), then additional ‘quality control’ checks may be made by the image analysis software to ensure that there are no gross abnormalities within the photograph that could compromise the result. Examples include highly uneven illumination or localised reflections. These maybe identified as marked gradients or locally atypical pixel intensities in the image of the body of the assay cassette. These abnormalities may be recognised because the body of the assay cassette should have a uniform colour and intensity. Deficiencies in the image may either be corrected by the software or an error message returned.

The knowledge of the position of the reaction chambers within a photograph of the assay cassette and the colour/brightness LUT may then be used to calculate values required for image analysis of the specimens (e.g. the intensity and colour of the local background).

The staining intensity of the specimens may then be determined (e.g. by corrected-difference in staining intensity between the specimen and local background).

Accordingly, this invention relates to methods, equipment or kits that assess the functional properties of complex biological materials prior to their use in regenerative therapies.

In particular, it relates to methods, equipment or kits that assess, under clinically appropriate time-scales and conditions, the functional properties of a representative sample obtained from a complex biological material, in order to predict if that material is of appropriate quality for employment in a cell, tissue or biomaterial-based therapy (e.g. a transplant procedure).

The present invention advantageously facilitates decision-making in a clinical environment. It is distinguished from most other available assays in that it combines (i) a short period of time from sample collection to result (i.e. 45-90 minutes); (ii) minimal/no additional capital equipment requirement; (iii) easy and error-free use by theatre staff under prevailing operating theatre conditions without the need for extensive additional training; (iv) inherently safe design and construction; (v) low cost; (vi) compatibility with current clinical practice; (vii) ease of waste disposal; and (viii) clarity of output.

In a specific embodiment, the present invention relates to methods, an apparatus and kits that assess the functional viability of a representative sample of human pancreatic islets isolated from a cadaveric human organ donor, prior to the possible transplantation of the remaining pancreatic islets into a recipient patient as a therapy for type I diabetes.

Accordingly, the present invention provides methods, an apparatus and kit to assess the functional viability of donor organ-derived pancreatic islets, within the time-frame and practical constraints of a clinical environment, prior to the islets transplantation into a recipient patient as a treatment for type 1 diabetes.

The functional viability of an islet sample is determined using a combination of histological dyes that reveal if the islets are able to respond to changes in nutrient availability with changes in metabolic activity. Results from the assay may be judged qualitatively by an observer and/or fully quantified by image analysis. This arrangement is designed to assist in clinical decision-making by allowing a clinician to make a rapid ‘eye-ball’ judgement of the assays outcome before fully quantified data becomes available. The assay of the present invention may be used advantageously to rapidly (within 90 minutes of collection) and accurately predict the suitability of an islet preparation for use in a transplant procedure. The rapidity by which results concerning the viability of cells, not only pancreatic islet but other cell types, can be obtained is of great clinical significance and provides a real contribution to the field and art.

In a further embodiment, the present invention relates to methods, an apparatus and kit to assess the functional viability of complex biological materials for use in therapies for liver disease. For instance, the invention may be used to assess the quality of livers or liver cells donated from sub-optimal donors.

In a further embodiment, this invention relates to methods, an apparatus and kit to assess the functional viability of complex biological materials for use in therapies for lung disease.

In a further embodiment, this invention relates to methods, an apparatus and kit to assess the functional viability of complex biological materials for use in therapies for kidney disease.

In a further embodiment, this invention relates to methods, an apparatus and kit to assess the functional viability of marrow samples used in the treatment of disease. At present bone marrow is widely collected (e.g. from iliac crest) for use in the treatment of haematological disorders. It is also possible that, in the future, marrow-derived mesenchymal stem cells will become widely used in the treatment of cartilage, bone, fat, tendon, ligament and dermal disorders. It is known that small variations in marrow collection technique can markedly alter the cellular composition of a marrow sample, complicating the interpretation of clinical outcomes. The present invention advantageously allows for the functional properties of a marrow sample to be rapidly assessed, thereby facilitating clinical decision-making.

In a further embodiment, the present invention relates to methods, an apparatus and kit to assess the functional viability of cell populations enriched in adipose-derived mesenchymal stem cells intended for use in the treatment of disease.

### Example 1

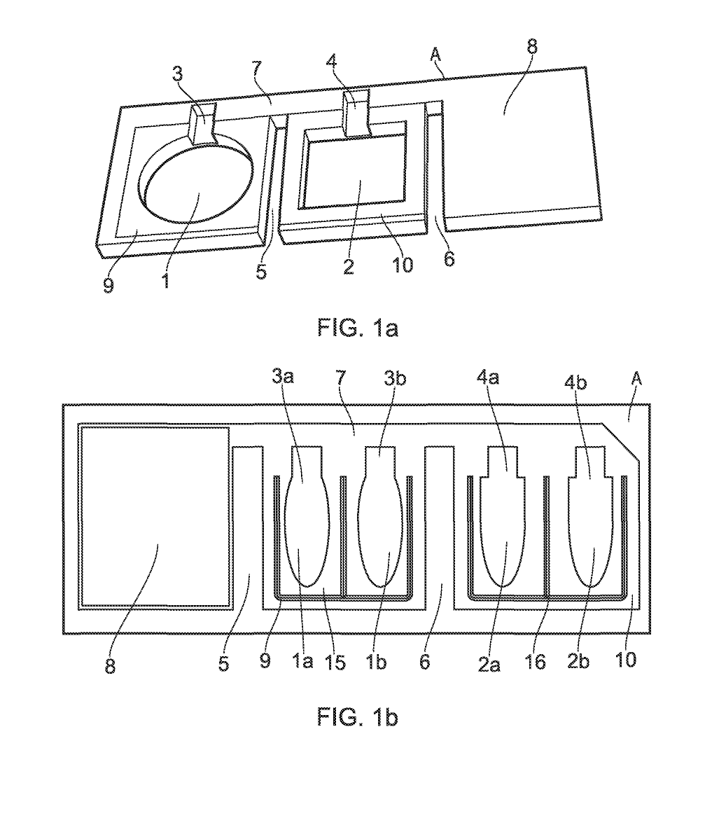

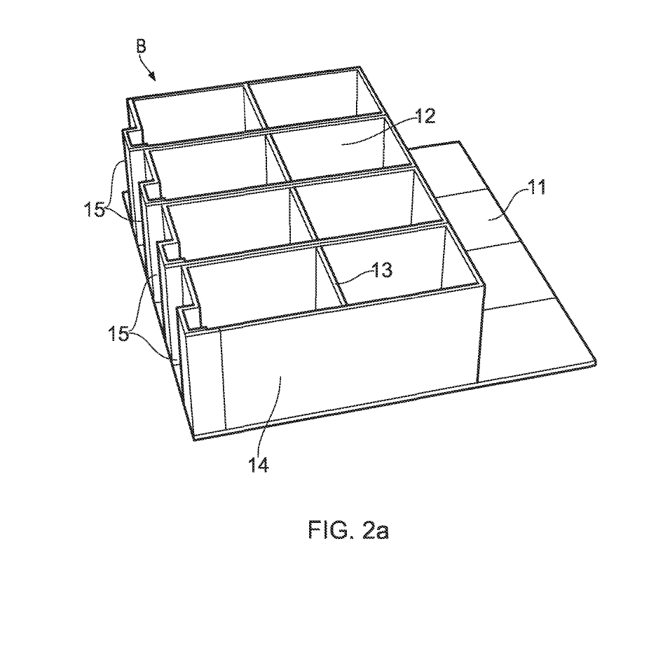

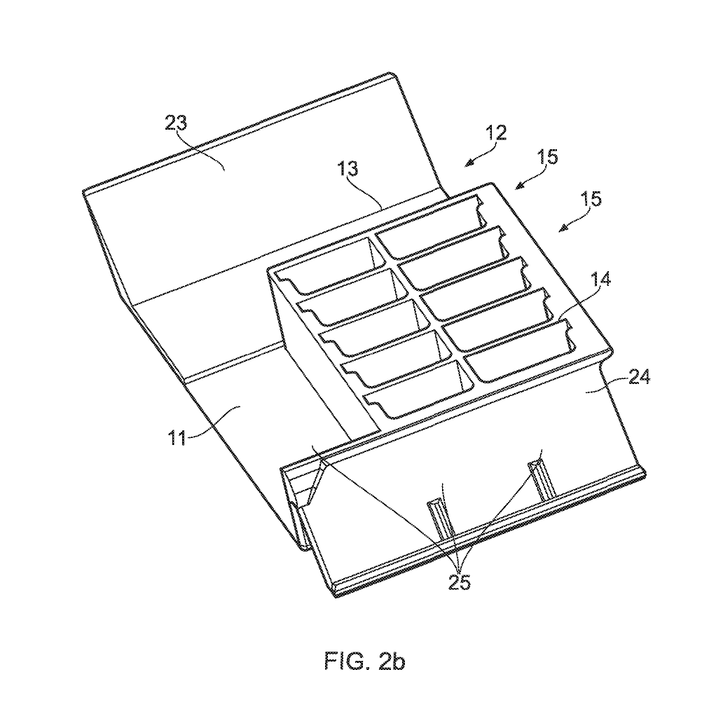

Two embodiments of the assay cassette of the invention are depicted in FIG. 1a and FIG. 1b. In addition, two embodiments of the reagent vessel of the invention are depicted in FIG. 2a and FIG. 2b.

### Assay Cassette

The assay cassette (A), as depicted in FIG. 1a, consists of two reaction chambers (1 and 2) attached to a base region (7) and contained within an enclosure or housing (9 and 10).

The assay cassette (A) as depicted in FIG. 1b, consists of two pairs of reaction chambers (1a, 1b; and 2a, 2b) attached to a base region (7) and contained within an enclosure or housing (9 and 10).

The reaction chambers may have a solid wall and a permeable barrier defining the internal area of the reaction chamber or alternatively two permeable barriers may define the walls of the internal area.

The particular embodiments of the assay cassettes of FIGS. 1a and 1b are suitable for use as a component in an assay for assessing the viability of donor organ-derived pancreatic islets. The two reaction chambers (1 and 2) (or two pairs of reaction chambers (1a, 1b; and 2a, 2b) have similar or equal volumes but differing shapes. Islet suspension is introduced into the reaction chambers via openings in the periphery of the enclosure (FIG. 1a: 3 and 4; FIG. 1b: 3a, 3b and 4a, 4b).

The external dimensions of the cassette and the asymmetric spacing of the two grooves in the cassette (5 and 6) only permit the assay cassette to be inserted into the reagent tray (or reagent vessel) (as depicted in FIG. 2a or 2b) in a single orientation thereby obviating human error during the process.

The assay cassette includes handling portion (8) which may be in the form of a multicoloured strip and which may serve as a reference or calibration standard for use when interpreting the results from the assay. Alternatively, the reference or calibration standard may be supplied separately.

The base region of the assay cassette (7) may include beading strips (FIG. 1b; 15 and 16) to allow a permeable barrier to be ultrasonically welded onto the base region.

The enclosure (i.e. assay cassette) has overall dimensions 25 mm×76 mm×3.5 mm (i.e. similar dimensions to a standard microscope slide) to facilitate observation of the cassette using a standard optical microscope and stage.

The two chambers (1 and 2) of FIG. 1a are of similar or equal volumes (e.g. 480 μl) but of differing shapes (e.g. square=test (e.g. High nutrient treated); round=control (e.g. low nutrient treated)). This arrangement facilitates the identification of which aliquot of the islet preparation was exposed to ‘test’ conditions (i.e. high nutrient medium or live cells) as opposed to ‘control’ conditions (i.e. low nutrient medium or dead ‘negative control’). This precaution minimises the risk of operator error when interpreting the assay result under stressful clinical conditions.

The two pairs of chambers (1a, 1b; 2a, 2b) of FIG. 1b are also of similar or equal volumes (e.g. 150 μl) but of differing shapes (i.e. round top=test; square top=control). This arrangement facilitates the identification of which aliquot of the islet preparation was exposed to ‘test’ conditions (i.e. high nutrient medium or live specimen) as opposed to ‘control’ conditions (i.e. low nutrient medium or dead ‘negative control’). This precaution minimises the risk of operator error when interpreting the assay result under stressful clinical conditions.

The assay cassette's enclosure is formed by injection moulding from a transparent thermoplastic (e.g. polystyrene, polycarbonate or poly methyl methacrylate) under clean conditions (Bradford Polymer CIC). Polystyrene is preferred because it offers superior resistance to damage when sterilised by gamma irradiation and acceptable properties for ultrasonic welding.

The regions of the enclosure wall through which the contents of the reaction chambers are observed may be approximately 500 μm thick.

An essentially two dimensional surface filter composed of a 60 μm nylon mesh covers the open face of the reaction chambers, permitting largely unrestrained reagent exchange into and out of the reaction chambers during staining (e.g. histochemical staining) but preventing cells (e.g. pancreatic islets) from leaving the chambers. The nylon filter can be ultrasonically welded onto the assay cassette. Appropriate welding conditions are: Energy 15J, Amplitude 75•m, Pressure 20 psi, Weld time 0.9 s, Distance 0.5 mm, Hold time 2 s using an industry standard 20 kHz Ultrasonic Welder (Sonics and Materials, Inc; Suffolk).

### Reagent Vessel

The reagent vessel or tray (B, FIG. 2a or FIG. 2b) comprises a series of interconnected but mutually independent reagent chambers (12) resting on a platform or base (11). The reagent vessel has a front wall (14) and the partitioning wall (13) is formed by adjacent side walls of each reagent chamber.

The external dimensions and shape of the assay cassette and reagent tray (in particular the spacing of the two grooves on the cassette (5 and 6) and the asymmetry in the reagent tray (FIG. 2a, item 15) only allow the assay cassette of FIG. 1a to be inserted into a reagent tray with the ‘square’ well on the left hand side and the ‘round’ well on the right hand side. This arrangement ensures that the islets in the square well are immersed into the high nutrient medium (the ‘test’ conditions) whilst the islets in the left hand well are immersed in the low nutrient medium (the ‘control’ conditions).

The external dimensions and shape of the assay cassette and reagent tray (in particular the spacing of the two grooves on the cassette (5 and 6) and the asymmetry in the reagent tray (FIG. 2b, item 15) only allow the assay cassette of FIG. 1b to be inserted into a reagent tray with the ‘round-topped’ wells on the left hand side and the ‘square topped’ wells on the right hand side. This arrangement ensures that the islets in the square topped wells are immersed into the ‘negative control’ medium whilst the islets in the round-topped wells are immersed in the ‘test’ medium.

Further asymmetries within the reagent tray (e.g. FIG. 2b, item 15) ensure that the assay cassette is placed into the reagent tray such that the biological samples in each of the reaction chambers of the assay cassette (FIG. 1b) receive equal and maximal exposure to the reagents.

Reagents are added to the reagent tray (FIG. 2a or FIG. 2b) and the reaction chambers may be sealed with a ‘tear-off’ lid consisting of a plastic laminated metal foil that is heat welded onto the chambers (e.g. at 165-175° C.; for 2.4 seconds). The filled and sealed reagent tray may then be sterilised by gamma irradiation.

Immediately prior to use the ‘tear-off’ lid is removed from the reagent tray.

### Assay

With regard to FIG. 1a, two aliquots of a pancreatic islet suspension (e.g. two ×450 μl aliquots) are introduced into the reaction chambers via openings in the periphery of the enclosure (3 and 4). The sample-loaded assay cassette is then inserted sequentially into rows 1-4 of the reaction tray (B, FIG. 2a) for, for example, 35, 6, 10 and 5 minutes respectively.

With regard to FIG. 1b, four replicate aliquots of a pancreatic islet suspension are introduced into the reaction chambers of the assay cassette via openings in the periphery of the enclosure (3a, 3b, 4a, 4b). The assay cassette may be placed on a ‘lectern’ at the rear of the reagent tray (23) during loading so that islets may be pipetted into the cassette ‘one-handed’, thereby simplifying use. This arrangement also reduces the risk of bubbles becoming trapped within the chambers of the assay cassette. The sample-loaded assay cassette is then inserted sequentially into the reagents contained within the chambers of rows 1-5 of the reaction tray for, for example, 5, 45, 6, 5 and 5 minutes respectively.

The assay cassette is supplied with a cover to seal the cassette after use, preventing liquid from spilling from the cassette during observation.

At the end of the staining process the colour of the sample in the reaction chambers is then compared by an observer. If the colour of the control and test samples are both the same, (i.e. approximately Pantone Cool Grey 8; red=150, green=148, blue=145; Hexadecimal #969491) then the islets appear to be non-functional, suggesting that the transplantation procedure should probably not proceed.

In contrast, if the colour of the control and test samples is different, with the ‘low nutrient’ or negative ‘control’ well(s) having a colour approximating to Pantone Cool Grey 8, whereas the ‘high nutrient’ or ‘test’ well(s) having a colour approximating to Pantone 7428 Dark crimson (i.e. red=109, green=45, blue=65; Hexadecimal #6D2D41; CMYK %, 0, 59, 40, 57), then the islets appear to be functionally viable. This result is consistent with islet transplantation proceeding.

The assay cassette may include a multicoloured strip on the handling portion (8). This acts as a reference standard for use during qualitative image analysis. One region of the strip is white (to enable an image processing programme to assess the colour balance and brightness of the illumination that has been used to observe the specimen). The other portion of the strip is coloured to simulate the staining of a strongly metabolically responsive islet and may be used as a positive control both by a human observer and by the image analysis software. A suitable positive control strip would be Pantone 7428 Dark crimson (i.e. red=109, green=45, blue=65; Hexadecimal #6D2D41; CMYK % 0, 59, 40, 57).

### Example 2a

An assay cassette and reagent tray as depicted in the embodiments of FIGS. 1a and 2a were used to assess the functional viability of a test pancreatic islet sample by comparing the colour change of a sample subjected to ‘high nutrient’ conditions to the colour change of a sample subjected to ‘low nutrient’ conditions.

The reagent tray (as depicted in the embodiment of FIG. 2a) consists of a multiwell plate comprising an orthogonal array of 8 cavities arranged as two columns and four rows. Although the cavities all have equal volumes, the asymmetry in the shape of the peripheral wall (15) ensures that the assay cassette (FIG. 1a) can only be inserted into the reagent tray in a single orientation. The cavities in the reagent tray are pre-loaded with the reagents required for the assay as shown in Table 1.

Reagents were prepared as follows. PBS and DMSO were obtained from Sigma. MTT (3-(4,5-Dimethylthiazol-2-yl)-2,5-diphenyltetrazolium bromide, a yellow tetrazole), is reduced to purple formazan in living cells, MTT (Sigma M5655) was dissolved (5 mg/ml) in PBS. Dithizone was dissolved (0.0025 g/ml) in DMSO. High Nutrient Medium (termed HNM) was prepared by mixing DMEM (500 ml; Sigma D5796) with 15% v/v foetal calf serum (Biosera), penicillin/streptomycin (5 ml; Sigma), L-glutamine (5 ml; Sigma) and non essential amino acids (5 ml; Sigma). Low Nutrient Medium (termed LNM) was prepared by diluting HNM 1 plus 5 with PBS (v/v).

TABLE 1
Reagents used in pancreatic islet functional viability assay.

| | Column 1. High | Column 2. Low | Incubation |
|---|---|---|---|
| Row | nutrient medium | nutrient medium | Time |
| 1 | HNM | LNM | 35 minutes |
| 2 | HNM supplemented | LNM supplemented | 6 minutes |
| | with 200 μl/ml MTT | with 200 μl/ml MTT | |
| | solution and 20 μl/ml | solution and 20 μl/ml | |
| | dithizone solution | dithizone solution | |
| 3 | 10% neutral buffered | 10% neutral buffered | 10 minutes |
| | formalin | formalin | |
| 4 | PBS | PBS | 5 minutes |

The assay cassette containing the pancreatic islets is then inserted sequentially into rows 1-4 of the reaction tray for 35, 6, 10 and 5 minutes respectively.

### Example 2B

This embodiment of the pancreatic islet functional viability assay compares the metabolic activity of duplicate islet samples cultured in high nutrient medium with duplicate islet samples cultured in low nutrient medium. Islets cultured in high nutrient conditions become metabolically active and stain with both MTT and dithizone. In contrast the samples cultured in low nutrient conditions become metabolically quiescent and remain comparatively unstained. The difference in staining is therefore a measure of the islets functional viability.

The reagent tray (as depicted in the embodiment of FIG. 2b) consists of a multiwell plate comprising an orthogonal array of 10 cavities arranged as two columns and five rows. Although the cavities all have equal volumes, the asymmetry in the shape of the tray and peripheral wall (15) ensures that the assay cassette (FIG. 1b) can only be inserted into the reagent tray in a single orientation. The cavities in the reagent tray are pre-loaded with the reagents required for the assay as shown in Table 2.

Reagents were prepared as described in Example 2A.

TABLE 2
Reagents used in pancreatic islet functional viability assay.

| | Column 1. High | Column 2. Low | Incubation |
|---|---|---|---|
| Row | nutrient medium | nutrient medium | Time (minutes) |
| 1 | PBS | PBS | 5 |
| 2 | HNM + D | LNM + D | 35 |
| 3 | MTT | MTT | 6 |
| 4 | 10% neutral buffered | 10% neutral buffered | 10 |
| | formalin | formalin | |
| 5 | PBS | PBS | 5 |

The assay cassette containing the pancreatic islets is then inserted sequentially into rows 1-5 of the reaction tray for 5, 35, 6, 10 and 5 minutes respectively.

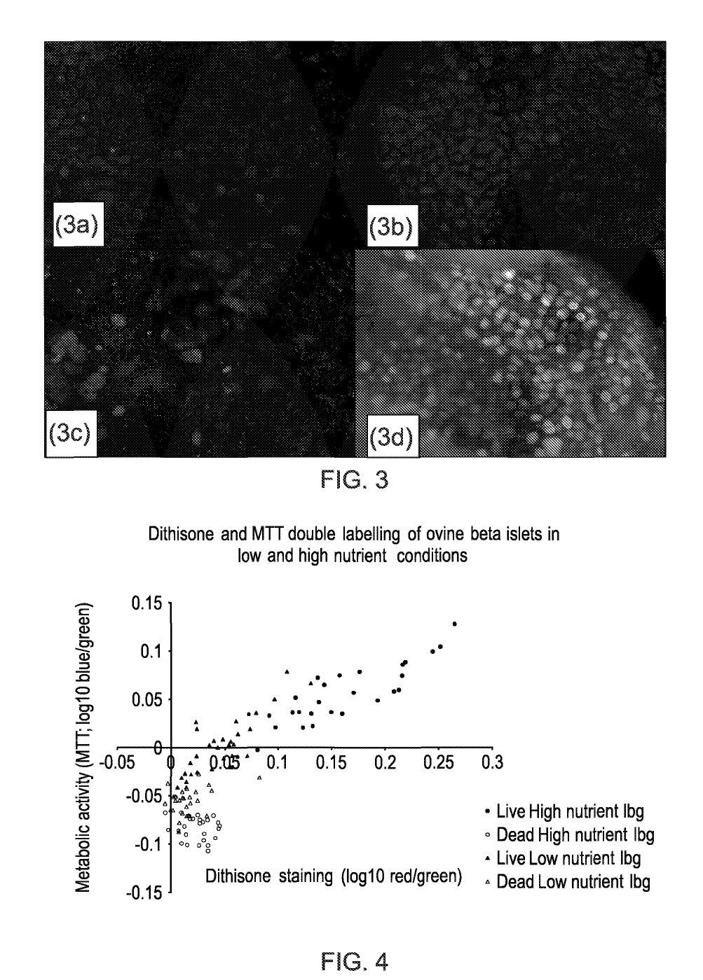

The results of the assay are shown in FIG. 3 and are described in more detail in Example 3.

### Example 3

The end-point of the assay of the invention may be assessed by both qualitative and fully-quantitative means of analysis.

Where the output from the assay is to be judged qualitatively, the results may simply be appraised by direct observation of the assay cassette. A coloured strip (e.g. Pantone 7428 Dark crimson) on the assay cassette may be used as a reference standard to assist in this process.

The results from the assay may be fully quantified by image analysis, either by direct measurement of the assay cassette. Alternatively the results may be quantified by analysis of recorded images (e.g. by photography or digital imaging).

The fully quantitative image analysis method depends upon the contrasting staining (e.g. histochemical staining) of (i) metabolically quiescent cells e.g. beta islet cells incubated in low nutrient medium; with (ii) metabolically active cells e.g. beta islet cells incubated in high nutrient medium.

Briefly, beta islet cells are glucose sensors. They respond to external glucose levels below 6 mM by becoming metabolically quiescent. In contrast they respond to external glucose levels above 6 mM by becoming metabolically active and then producing and secreting the glucose-regulating hormone insulin.

Biochemically, the glucose-triggered switch from metabolic quiescence to metabolic activity is mediated by (i) glucokinase-dependent increases in glucose phosphate production; (ii) enhanced mitochondrial activity (including raised levels of mitochondrial phosphoenol pyruvate) and, (iii) highly increased levels of cytosolic NAD(P)H.

Histochemical redox dyes that reveal high levels of mitochondrial activity and/or cytosolic NAD(P)H can therefore be used to identify living beta cells that are responding to glucose levels above 6 mM with increased mitochondrial activity. Suitable dyes include MTT which forms an insoluble blue formazan product when a cell's mitochondria are metabolically active. From an image processing perspective blue objects have more intense blue channel intensities than green channel intensities (i.e. log₁₀ blue/green >0). In contrast, unstained, MTT negative cells (e.g. dead cells or living islet cells cultured in medium containing <6 mM glucose), have approximately equal blue and green channel intensities (i.e. log₁₀ blue/green ˜0). Metabolically active cells therefore have higher log₁₀ blue/green. The numerical difference between log₁₀ blue/green for beta islets equilibrated in medium containing (i) high glucose and (ii) low glucose is therefore a measure of the cells ability to respond to increased glucose availability with increased levels of metabolic activity. The advantage of using this parameter instead of simply measuring the blue channel intensity is that it is largely independent of the overall illumination intensity, staining intensity, white balance and local optical density of the specimen. In contrast all of these variables influence the value of the blue channel alone.

In parallel, functionally viable beta islet cells that are responding to increased glucose produce and secrete increased levels of the hormone insulin. Insulin exists within beta cells as a complex with zinc. Insulin producing cells therefore have higher levels of zinc compared to other cell types. Intercellular zinc can be visualised using the membrane permeant, zinc-chelating dye, ‘dithizone’, which stains zinc-rich cells pink. Dithizone staining can therefore be used to distinguish between insulin rich beta cells and other pancreatic cell types. Furthermore, because dying beta cells tend to degranulate and shed their insulin into the medium, dithizone tends to stain viable beta cells more intensely than dead/dying beta cells. From an image processing perspective pink objects have log₁₀ (red/green)>0. This parameter can therefore be used in dithizone stained pancreatic samples to identify viable beta islet cells.

To identify glucose responsive, functionally viable beta islet cells therefore, test aliquots of an islet suspension can be pre-incubated in either low or high nutrient conditions for sufficient time that their metabolism can adapt to the prevailing nutrient availability. The islets can then be double labelled with a combination of dithizone and MTT. Results indicate that only the viable islets incubated in high glucose medium stain with both dyes. FIG. 3 shows a monochrome representation of live and dead (formalin fixed) ovine pancreatic islets incubated in either low or high nutrient media and stained simultaneously with both dithizone and MTT. Results indicate that only the viable islets incubated in high nutrient medium stained strongly with both dyes (bright pixel intensities). In contrast, dead islets and/or islets incubated in low nutrient medium remained unstained (dark pixel intensities). FIG. 3a shows dead islets in low nutrient media. FIG. 3b shows dead islets in high nutrient media. FIG. 3c shows live islets in low nutrient medium. FIG. 3d shows live islets in high nutrient medium. The pink-purple staining of the MTT and dithizone labelled specimens was converted into a monochrome image by manipulating the pixel intensities in the digital images according to the formula ((blue-green)+(red-green)/2) using Paint Shop Pro 7.

### Example 4

Live and dead ovine pancreatic islets were incubated in either low or high nutrient media and stained with both dithizone and MTT. The parameters log₁₀ (blue/green) (a measure of metabolic activity as indicated by MTT staining) and log₁₀ (red/green) (a measure of insulin content as indicated by dithizone staining) were highest for viable islets incubated in high nutrient medium and lowest for the dead islets. Viable islets in low nutrient medium gave intermediate results. FIG. 4 shows that double labelling with MTT and dithizone is reflected by the predicted changes in the parameters log₁₀ (blue/green) and log₁₀ (red/green), with viable islets incubated in high glucose medium showing the highest values for both these two parameters.

### Example 5

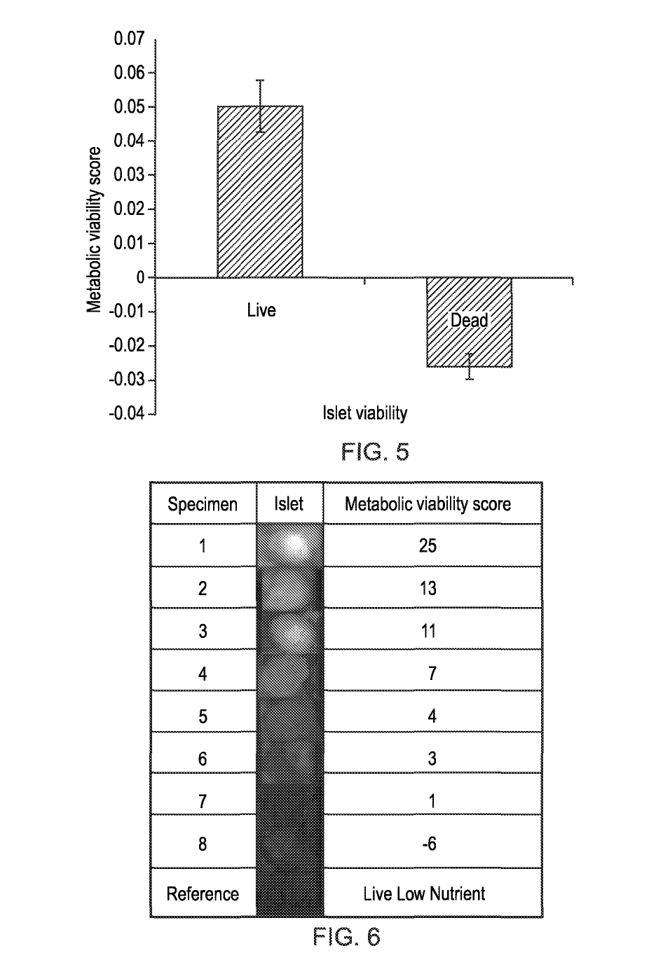

The parameter log₁₀ (blue/green)_{High glucose}−log₁₀ (blue/green)_{Low glucose} was calculated for live and dead ovine pancreatic islets. FIG. 5 illustrates that the value of the parameter log₁₀ (blue/green)_{High glucose}−log₁₀ (blue/green)_{Low glucose} can be used as a convenient ‘metabolic viability score’ to reflect islet viability.

FIG. 6 shows a selection of images of individual islets cultured in high nutrient medium and then double labelled with dithizone and MTT. The value of the parameter (log₁₀ (blue/green)_{High glucose}−mean (log₁₀ (blue/green)_{Low glucose})×100 can be used to assess an individual islet's functional viability when it is cultured under high nutrient conditions. Using this scoring system a value of 25 indicates a highly double labelled ovine islet (i.e. an insulin-rich, glucose responsive islet) whereas dead ovine islets have scores <=1. The pink-purple staining of the MTT and dithizone labelled specimens was converted into a monochrome image by manipulating the pixel intensities in the digital images according to the formula ((blue-green)+(red-green)/2) using Paint Shop Pro 7. Results suggest that for an individual islet in high nutrient medium, the value of the parameter (log₁₀ (blue/green)_{High glucose}−mean (log₁₀ (blue/green)_{Low glucose})×100 can be used to assess an individual islet's functional viability. (In this scoring method an average value is determined for the islets in the low nutrient conditions and this value is then used as a standard when scoring individual islets cultured in high nutrient conditions). Under this scoring system a value of 25 indicates a highly double labelled islet (i.e. an insulin-rich, glucose responsive islet) whereas dead islets have scores <=1.

The absolute scores for individual islets growing in high nutrient medium, the distribution of these scores and the mean value for the population reflects the functional viability of an islet preparation. This data may be used to assist in determining if an islet preparation is suitable for clinical use in an islet transplantation procedure.

### Example 6

Experiments were performed to establish the assay conditions used to evaluate pancreatic islets with this invention.

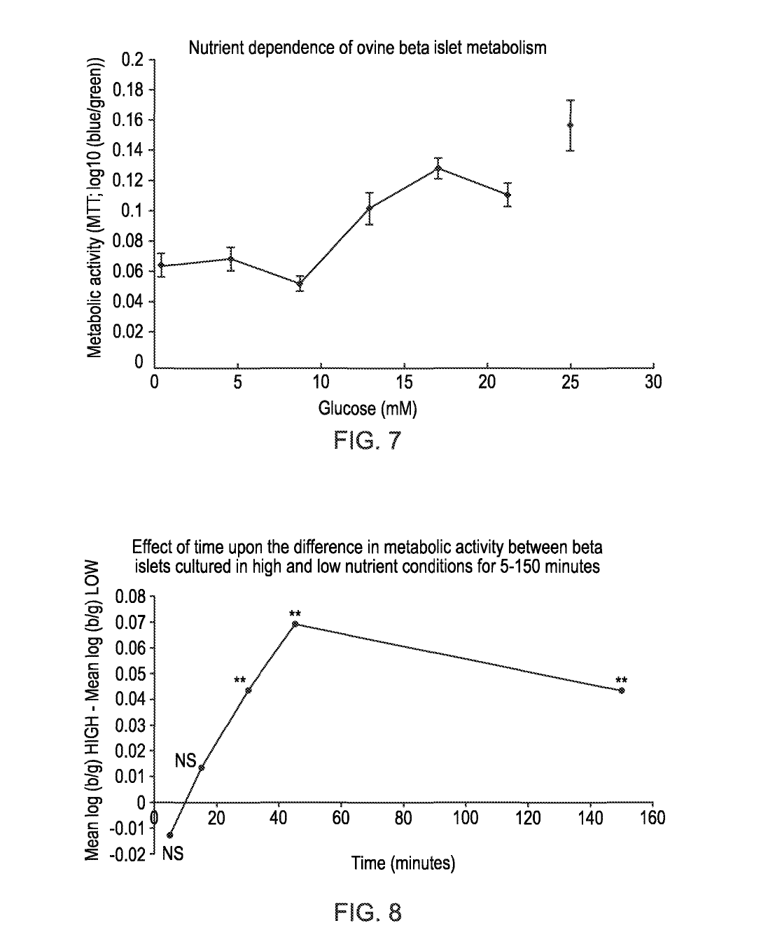

In order to determine the threshold concentration of nutrients required to switch islets from a quiescent to a metabolically active state, islets were first rendered quiescent by nutrient depletion by pre-incubation in very low nutrient media (0.4 mM glucose; 3 h). The cells were then transferred to medium containing higher levels of nutrients (0.4 mM-20 mM glucose for 2 h) before being double labelled with MTT and dithizone. FIG. 7 shows that cells incubated in >10 mM glucose for the second incubation period responded to the increased nutrient levels with a statistically significant increase in metabolic activity. It was therefore decided to use 1 mM glucose as the ‘low’ nutrient conditions and 25 mM glucose as the ‘high’ nutrient conditions in the assay. Islets incubated throughout the experiment in 25 mM glucose (blue) served as controls.

In order to determine the minimum length of time that islets must be exposed to altered levels of nutrients before they will show a statistically significant change in metabolic activity, islets were transferred from high to low nutrient conditions for 5 to 150 minutes and their labelling compared to cells maintained in high nutrient conditions throughout the experiment. FIG. 8 reveals that the islets require 30-45 minutes to show a statistically significant response to changes in nutrient availability in vitro. This shows that, in the assay, islets will have to be pre-incubated in low and high nutrient medium for at least 30 minutes before staining if a metabolic response to nutrient availability is to be observed.

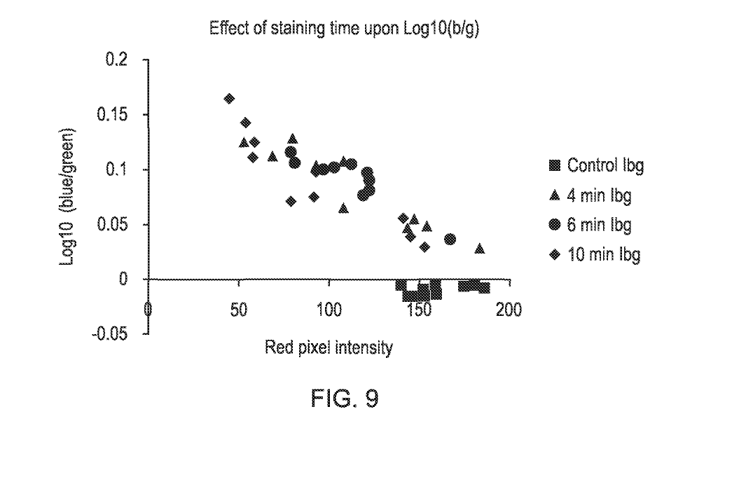

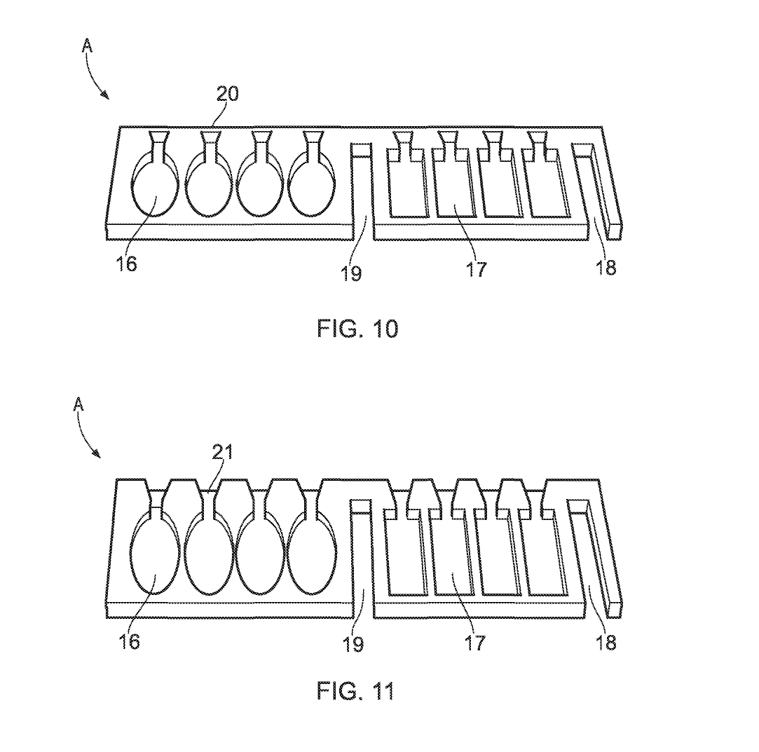

In order to determine the optimal duration for staining, islets were incubated in MTT for 4 to 10 minutes and log₁₀ blue/green calculated for individual islets. FIG. 9 indicates that staining durations between 4-10 minutes give comparable log₁₀ (blue/green) values, suggesting that small errors in staining duration are unlikely to compromise the assay.

### Example 7

The assay cassette shown in FIG. 1a has only a single reaction chamber for the ‘treated’ and ‘control’ samples. This arrangement is preferable for routine clinical situations where sample availability is a concern. However, for more research based applications, or situations where sample availability is not a concern, the assay cassette A shown in FIG. 1b or FIG. 10 may be preferable since they offer the possibility of making duplicate measurements. The assay cassette shown in FIG. 10 is particularly well suited to research applications where sample availability is not a limiting consideration. In this embodiment of the invention the reagent chamber housing can accommodate several reaction chambers (16, 17) having septal ports (20) within the same enclosure or reaction chamber housing and the asymmetric spacing of the two grooves in the cassette (18 and 19) only permit the assay cassette to be inserted into the reagent tray (as depicted in FIG. 2) in a single orientation thereby obviating human error during the process.

### Example 8

For quality control assays for solid organs, (e.g. liver) it may be preferable to assess multiple needle biopsies of the tissue because the viability of the organ may differ between different sites. Furthermore, the geometry of the biopsy collection needle precludes the use of the assay cassette shown in either FIG. 1 or FIG. 10. Accordingly, the assay cassette shown in FIG. 11 allows multiple needle biopsies to be collected and placed into the reaction chambers for assessment. This particular embodiment is particularly well suited for measuring multiple, duplicate needle biopsies from ‘marginal quality’ donated organs. This embodiment allows multiple needle biopsies to be collected and placed into the reaction chambers (16 and 17) via openings (21) for assessment.

### Example 9

The reagents described in Example 2A and 2B are optimised for staining performance rather than shelf-life. However, it is recognised that within a clinical environment the opportunity to tolerate more flexible and prolonged storage conditions could be beneficial. Accordingly, this reagent formulation permits prolonged storage at variable temperatures (>0° C.). The cavities in the reagent tray (as depicted in the embodiment of FIG. 2a or 2b) are pre-loaded with the reagents required for the assay as shown in Table 3.

Reagents are prepared as follows. PBS and DMSO are obtained from Sigma. Dithizone is dissolved (0.05 g/ml) in DMSO (5 ml). MTT (Sigma M5655; 500 mg) is dissolved (1 mg/ml) in 500 ml PBS. High nutrient medium (HNM) is prepared by mixing DMEM (Sigma D1145; 500 ml) with 75 ml HIFCS (BioSera) and non-essential amino acids (Sigma M7145; 5 ml). Low nutrient medium (LNM) is prepared by mixing HNM (8 ml) with PBS (492 ml). Phenol red (Sigma P0290; 1.59 ml) and dithizone stock solution (0.5 ml) is then added to 500 ml of both the HNM and LNM.

These reagents are added to the reagent tray as shown in table 3.

TABLE 3
Long shelf-life reagents used in the
pancreatic islet functional viability assay.

| | Column 1. High | Column 2. Low | Incubation |
|---|---|---|---|
| Row | nutrient medium | nutrient medium | Time |
| 1 | HNM | LNM | 35 to 40 |
| | | | minutes |
| 2 | MTT | MTT | 6 |
| | | | minutes |
| 3 | 10% neutral buffered | 10% neutral buffered | 10 |
| | formalin | formalin | minutes |
| 4 | PBS | PBS | 5 |
| | | | minutes |

The assay cassette containing the pancreatic islets is then inserted sequentially into rows 1-4 of the reaction tray for a sufficient incubation time e.g. for 35 to 40, 6, 10 and 5 minutes respectively.

### Example 10

An assay cassette and reagent tray as depicted in the embodiments of FIGS. 1b and 2b were used to assess the functional viability of duplicate pancreatic islet ‘test’ samples by comparison to duplicate devitalised negative control islet samples.

The reagent tray (as depicted in the embodiment of FIG. 2b) consists of a multiwell plate comprising an orthogonal array of 10 cavities arranged as two columns and five rows. Although the cavities all have equal volumes, the asymmetry in the shape of the tray and peripheral wall (15) ensures that the assay cassette (FIG. 1b) can only be inserted into the reagent tray in a single orientation. The cavities in the reagent tray are pre-loaded with the reagents required for the assay as shown in Table 4.

Reagents were prepared as follows:

PBS, glucose, non-essential amino acid mix, MTT (3-(4,5-Dimethylthiazol-2-yl)-2,5-diphenyltetrazolium bromide, a yellow tetrazole dye that is reduced to purple formazan in living cells), neutral buffered formalin, dithizone and DMSO were obtained from Sigma.

70% ethanol was prepared by mixing ethanol and water 70:30 v/v. MTT was dissolved (1 mg/ml) in PBS. Dithizone was dissolved (0.0025 g/ml) in DMSO.

High Nutrient Saline plus dithizone (termed HNS+D) was prepared by mixing PBS (500 ml) with glucose (2.25 g), non-essential amino acid mix (5 ml) and dithizone solution (1.5 ml).

TABLE 4
Reagents used in a pancreatic islet functional viability assay
that compares the metabolic activity of test samples of
islets with devitalised negative control islet samples.

| | Column 1. | Column 2. | Incubation |
|---|---|---|---|
| Row | Test medium | Negative control | time (min) |
| 1 | PBS | 70% ethanol | 5 |
| 2 | HNS + D | HNS + D | 45 |
| 3 | MTT | MTT | 5 |
| 4 | NBF | NBF | 5 |
| 5 | PBS | PBS | 5 |

As discussed in Example 3, pancreatic islets are nutrient sensors that respond to high nutrient levels with high levels of metabolic activity (particularly high levels of mitochondrial activity) and subsequent insulin production. The insulin is stored in vesicles prior to release as a complex with zinc ions. In contrast, islets in low nutrient conditions become metabolically quiescent and show low levels of insulin production. Furthermore, dead or dying islets tend to degranulate, shedding their insulin into the medium. As a consequence, highly metabolic islets stain with both the metabolic dye MTT (blue) and the zinc chelating dye dithizone (pink) to produce a dark purple colour. In contrast, dead islets show minimal levels of MTT and dithizone staining irrespective of their nutrient environment.

Previous results have shown that exposing clinical or laboratory-derived islet preparations to high nutrient conditions for 45 minutes in vitro is sufficient to induce an elevated metabolic response in even the most quiescent of cells (see Example 6).

Staining after 45 minutes in high nutrients therefore reflects the cells ability to respond physiologically and is therefore a measure of their functional viability.

The nutrients (glucose and amino acids) are supplied in a phosphate buffered saline solution rather than in the more usual bicarbonate buffered cell culture medium to allow the assay to be performed at room temperature and without the need for a CO₂ gassed incubator.

In this example, devitalised islets (killed with 70% ethanol) serve as a negative control during visual inspection and to assist in calibration during image processing.

The assay cassette allows duplicate test and control samples to be assessed, minimising the chance the result will be compromised by sampling errors.

Overall, this approach gives the clearest result (and is preferred by clinicians).

### Example 11

At the end of the staining process discussed in Example 10 the colour of the samples in the two pairs of wells is compared.

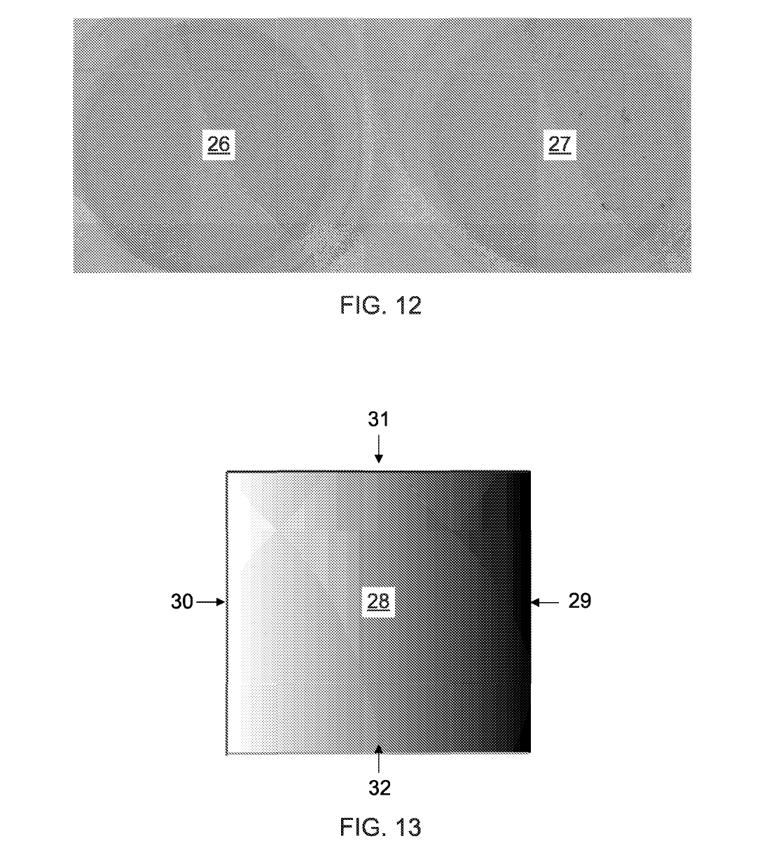

The result of the assay may be assessed qualitatively by observation with the naked eye. A viable islet sample is denoted by the appearance of dark purple—blue black dots within the test chambers (FIG. 12 item 27) whereas the control chambers remains devoid of such darkly stained particular material (FIG. 12 item 26). In contrast, if the appearance of the two pairs of wells is essentially the same (i.e. no darkly stained particulate material in either the test or control pairs of wells) then a negative result is indicated (i.e. the islet sample was non-viable).

### Example 12

The result of the assay (for instance as described in Example 10) may be quantified using a digital camera and software may be supplied with the assay kit. The stained assay cassette is placed upon the lectern at the front of the reaction vessel (FIG. 2b, 24) and photographed using a standard digital camera. Asymmetries in the reagent tray (25) ensure that the assay cassette may only be placed onto the lectern (24) in the correct orientation.

The software uses a calibration reference image located on the handling portion of the assay cassette (FIG. 1b, item 8) to calibrate and standardise the image in terms of brightness, orientation, linear dimensions and colour balance. FIG. 13 shows a suitable calibration reference image (size 20 mm×25 mm) consisting of 256 vertical grey stripes of decreasing intensity (ranging from white to black; FIG. 13 item 28). The central grey area is surrounded on the right side by a black line (29), on the left by a red line (30), at the top by a blue line (31) and at the bottom by a green line (32). These features are recognised by the software and are used, for instance, to determine the midline of the assay cassette in a photograph and calculate the number of pixels per mm in the image. From this information the approximate position of the test and control chambers in the photograph may be predicted.

Once the overall features of the cassette have been identified within the photograph, quality control tests may be performed automatically on the image by the software (e.g. to ensure that (i) the cassette has been photographed approximately horizontally and at the centre of the image and (ii) that the image is not very unevenly illuminated). If the image passes these tests then staining properties of the islets may then be determined by comparing individual pixel intensities with local backgrounds and the grey values of the calibration reference image.

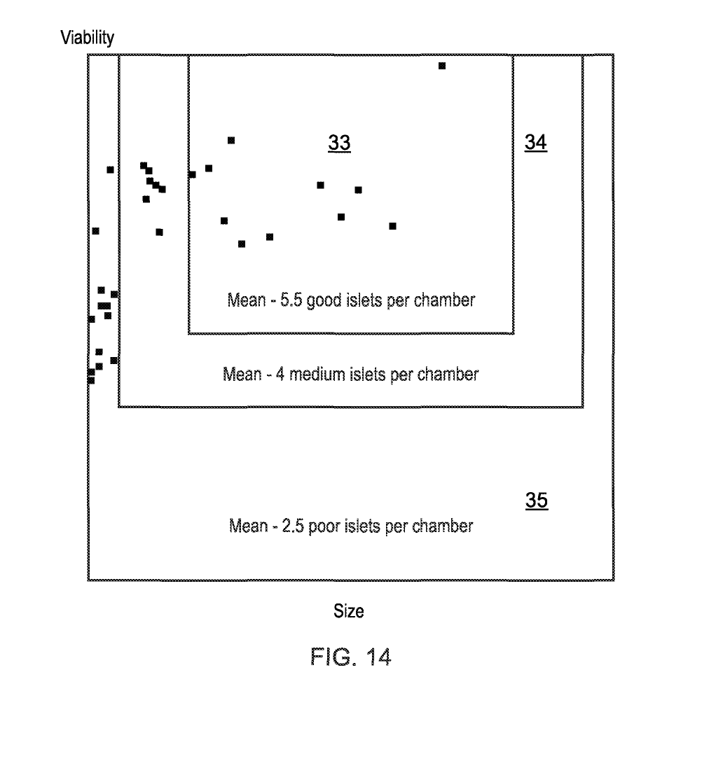

The output of the software quantifies the morphology and viability of the islets within a sample (e.g. number of islets in a sample, their individual sizes and staining intensities, together with information on the total staining levels within the sample). These parameters are then summarised as a ‘scattergram’ to assist rapid clinical interpretation (FIG. 14). This scattergram is constructed by superimposing a dot depicting the size and metabolic activity of an individual islet from a sample upon a background image showing the expected range of values for ‘good’ islets (green; item 33), ‘mediocre’ islets (orange; 34) and poor islets (red; 35). Background staining (i.e. if there are any darkly staining particles in the negative control) may also be displayed on the scattergram.

The numerical data generated by the software is tabulated automatically and may be exported to a spreadsheet (e.g. Excel).

### Example 13

The results from the staining method and image processing software were verified by comparing them with an established laboratory method for assessing cell viability. Mixtures of viable and devitalised human fibroblasts ranging from 0% viable to 100% viable were stained with dithizone (30 micrograms/ml) and MTT (1 mg/ml). Samples of the stained cell mixtures (100×10³ cells) were extracted with acid isopropanol (16 h) and the absorption measured at 570 nm using a spectrophotometer.

Further samples of the MTT and dithizone stained mixtures of viable and devitalised cells (10×10³ cells, i.e.˜the number of cells in 10 islets) were centrifuged (5000 rpm; 3 min) in Eppendorf tubes to produce an elongated pellet on the side of the tube.

The pellets were photographed using a Panasonic Lumix FZ45 digital camera set to its manual zoom function and the resulting images analysed using the algorithms described in example 12.

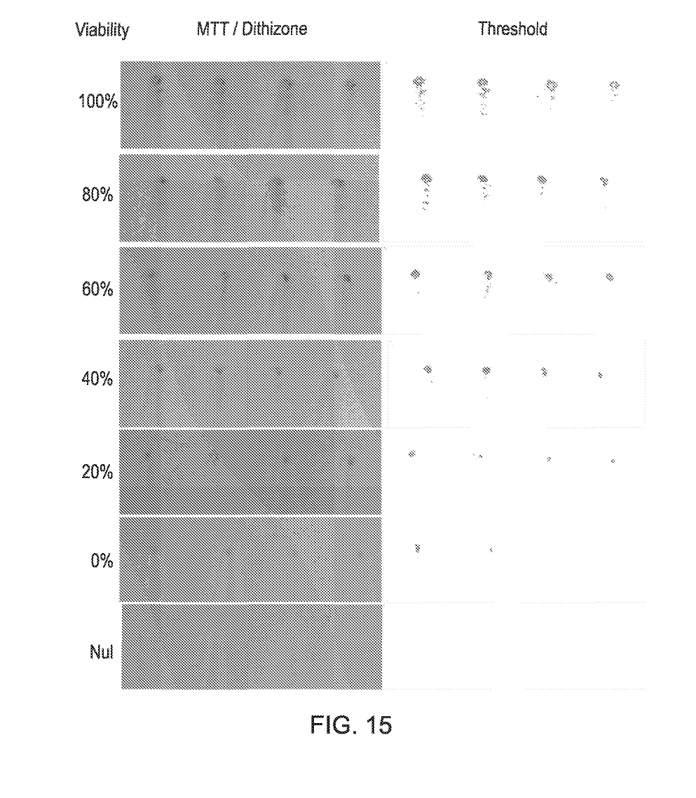

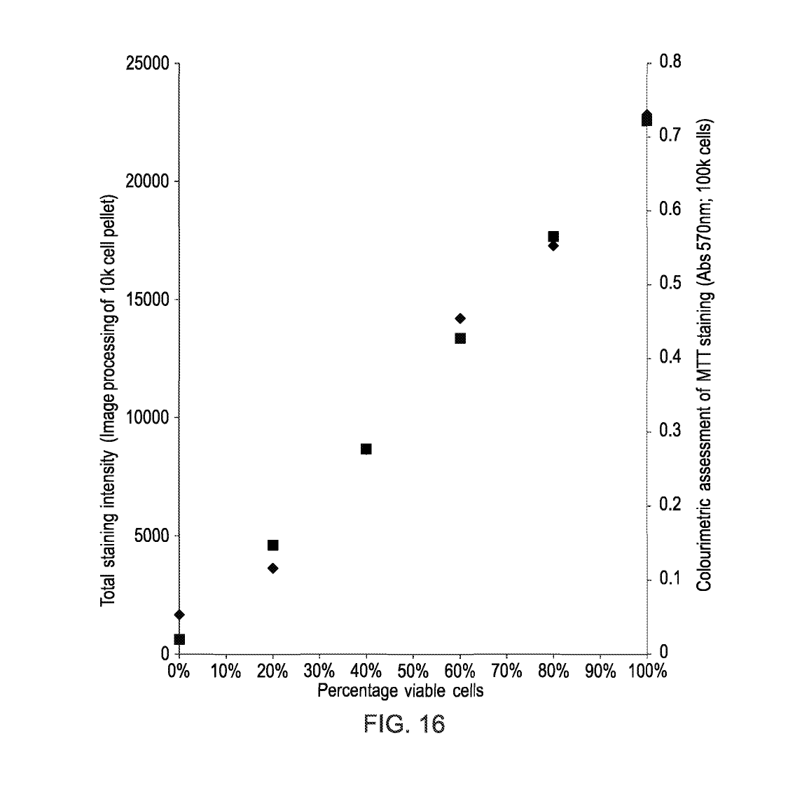

Results showed (FIG. 15) that the software could correctly distinguish the stained cells from the surrounding background image. FIG. 16 shows that the software enables the percentage viability of the cells to be determined over the same range as the established biochemical method.

### Example 14

A common cause of a loss of viability in transplant materials is anoxia followed by re-oxygenation. Human fibroblasts and Min-6 pancreatic islet-like cells were cultured for 16 h at 37° C. in DMEM supplemented with 10% foetal calf serum in an atmosphere of either (i) humidified air containing 5% CO₂ or (ii) a humidified hypoxic atmosphere containing 1% oxygen and 5% CO₂. The cells were then returned to normoxic conditions for a further 45 minutes (i.e. humidified air containing 5% CO₂ at 37° C.). The cells exposed to hypoxia and re-oxygenation showed profound distress within 15 minutes whereas the control cultures appeared morphologically normal.

The two cell populations were stained with dithizone (30 micrograms/ml) and MTT (1 mg/ml), trypsinised and the released cells combined with any non-adherent cells from the culture medium. Aliquots (10×10³ cells) were then centrifuged in Eppendorf tubes, the supernatant discarded and the pellets photographed and image processed.

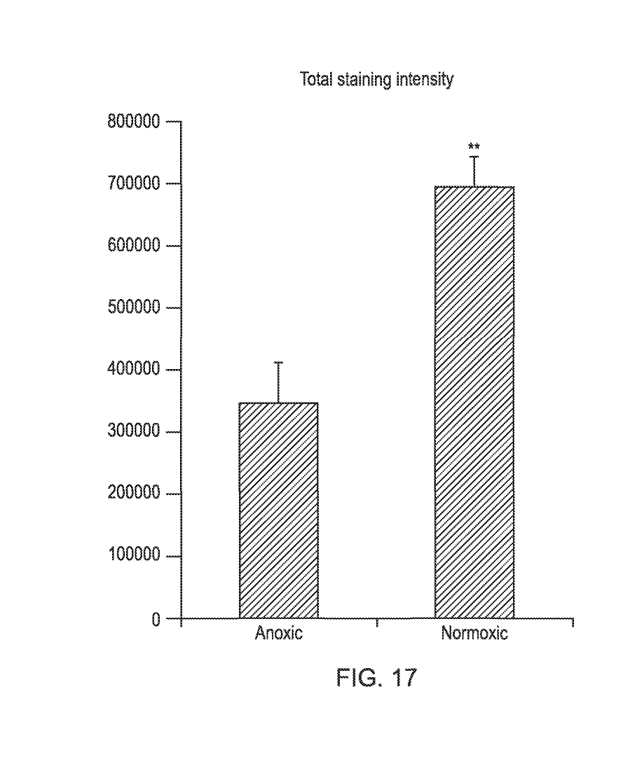

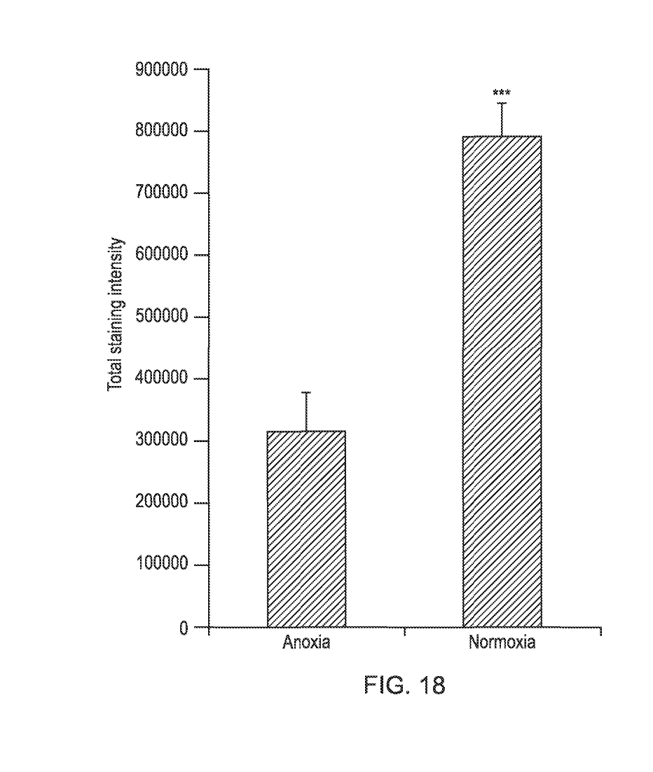

FIGS. 17 and 18 show that transient hypoxia produced a statistically highly significant decrease in staining intensity (i.e. viability) compared with normoxic controls for both the fibroblasts and Min-6 cells.

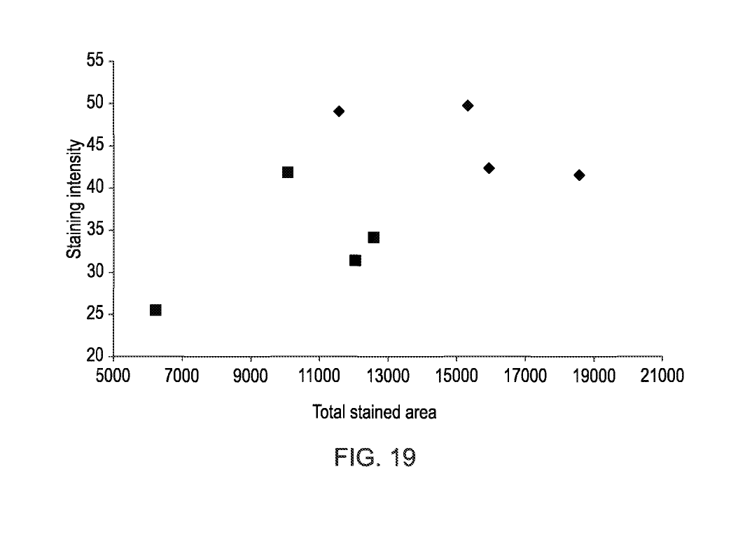

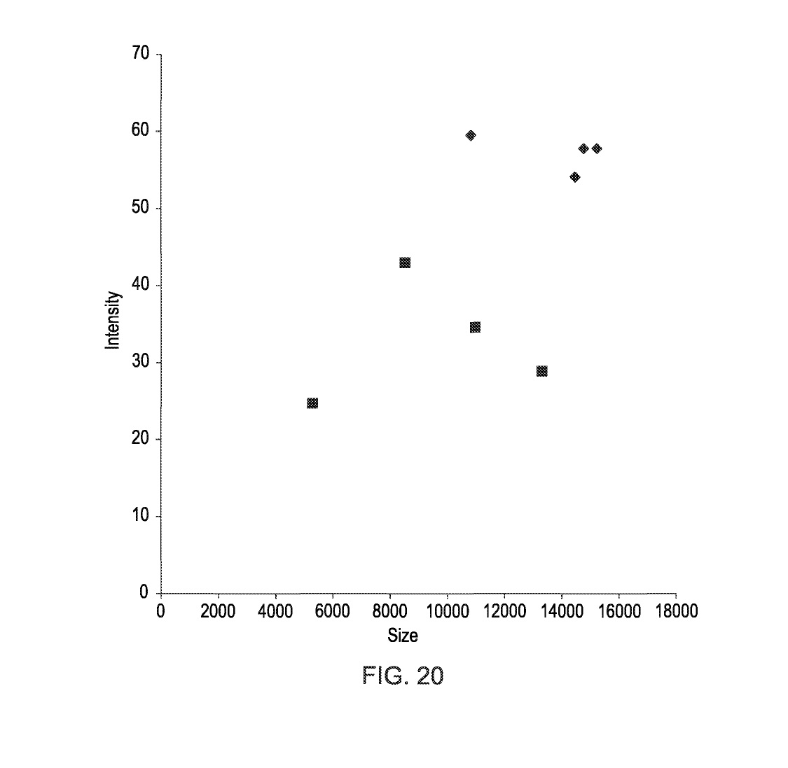

FIGS. 19 and 20 show that the results from individual pellets may be displayed on a scattergraph of staining intensity vs size as shown in FIG. 14.

### Example 15

The assay cassette is approximately the same size as a standard microscope slide enabling it to be conveniently examined in detail by light microscopy.

Islets stained with dithizone and MTT were assessed qualitatively by visual inspection. They were then assessed semi-quantitatively using a reference colour chart. Viable islets display a colour approximating to Pantone 7428 Dark crimson (i.e. red=109, green=45, blue=65; Hexadecimal #6D2D41; CMYK % 0, 59, 40, 57) whereas non-viable islets display a colour approximating to Pantone Cool Grey 8, (i.e. red=150, green=148, blue=145; Hexadecimal #969491)

The staining intensity of individual islets viewed by microscopy within the assay cassette may be further assessed by image processing. For instance parameter log₁₀ (blue/green)_{test islet}−mean log₁₀ (blue/green)_{negative control} islets may be used as a convenient ‘metabolic viability score’ to reflect islet viability (see example 5 and FIGS. 5 and 6). Optionally, the ‘metabolic viability score’ may be multiplied by 100 to give a ‘whole number’ answer in order to facilitate communication (FIG. 6).

### Example 16

Dithizone stock solution was prepared by dissolving dithizone (5 mg) in DMSO (5 ml). MTT (1 mg/ml) was dissolved in PBS.

Neonatal rat liver was isolated immediately post mortem, chopped into small fragments and cultured in DMEM/FCS for 0 to 135 minutes. Additional samples of neonatal rat liver were killed by briefly boiling in water in a microwave (negative controls).

To measure the viability of the samples, aliquots of the liver fragments were placed in Falcon 70 μm nylon cell strainers and sequentially (i) rinsed in PBS; (ii) stained with dithizone (3 μl dithizone stock solution per ml; 6 minutes), (iii) reacted with MTT (1 mg/ml in PBS; 8 minutes); (iv) fixed in neutral buffered formalin; and (v) rinsed in PBS.

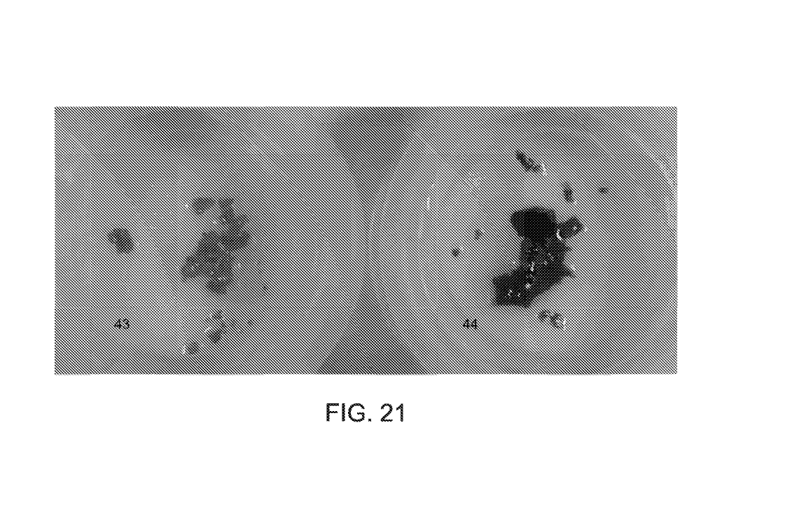

FIG. 21 shows a monochrome image of dead liver fragments (43) and live liver fragments (44) stained with dithizone and MTT immediately post mortem. The dead tissue stained light brown whereas the live tissue stained purple.

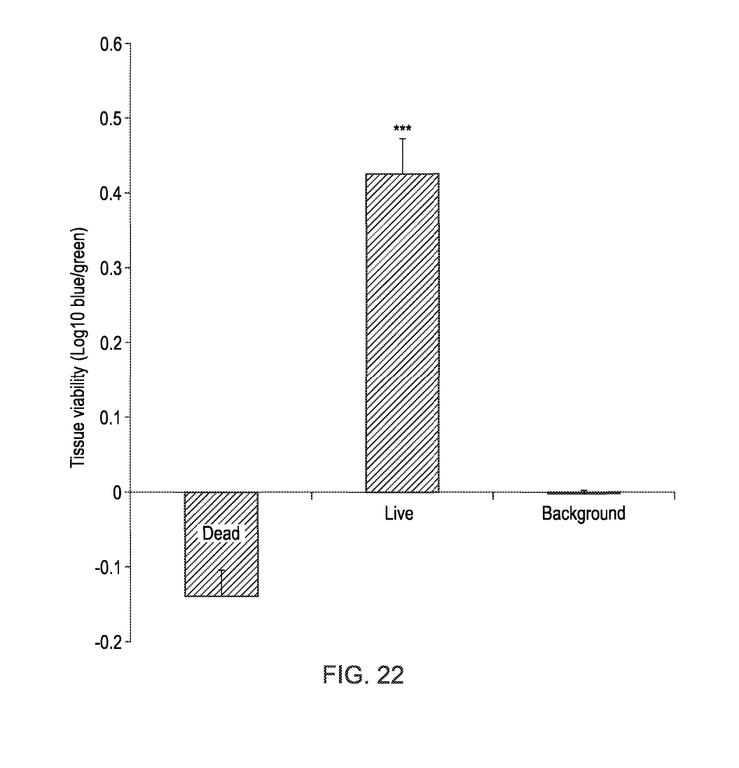

FIG. 22 shows the stained liver tissue quantified by image processing using the parameter Log 10 (blue channel pixel intensity/green channel pixel intensity) to distinguish between viable and non viable tissue.

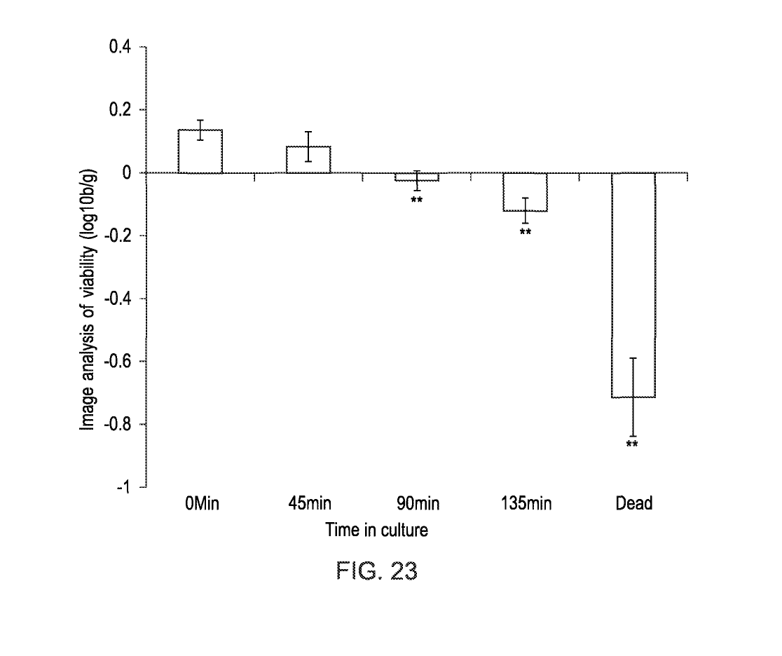

FIG. 23 shows that the image processing parameter Log 10 (blue channel pixel intensity/green channel pixel intensity) can be used to assess the loss of tissue viability during storage. In these experiments a storage period of 45 minutes produced a discernable drop in staining intensity. This loss in staining was statistically highly significant after 90 minutes. Staining was indistinguishable from the dead negative control samples after storage for 16 hours.

In addition, results showed that liver samples that had been incubated in hypoxic conditions for 45 minutes (1% O₂ and 5% CO₂) and then re-oxygenated in air for 15 minutes displayed reduced staining intensity compared to samples that had been incubated in normoxic conditions for 60 minutes. This is significant as transient hypoxia during storage is a major cause of diminished graft viability.

### Example 17

The assay cassette (as depicted in the embodiment of FIG. 11) was used to assess the functional viability of biopsies of liver tissue (biopsies harvested using a Tru-Cut 14G×7.6 cm needle; Cardinal Health). Devitalised liver biopsies were used as negative controls.

The reagent tray consists of a multiwell plate comprising an orthogonal array of 10 cavities arranged as two columns and five rows.

Although the cavities all have equal volumes, the asymmetry in the shape of the tray and peripheral wall ensures that the assay cassette (FIG. 11) can only be inserted into the reagent tray in a single orientation. The cavities in the reagent tray are pre-loaded with the reagents required for the assay as shown in Table 5.

Reagents were prepared as follows.

PBS, MTT (3-(4,5-Dimethylthiazol-2-yl)-2,5-diphenyltetrazolium bromide, a yellow tetrazole dye that is reduced to purple formazan in living cells), neutral buffered formalin, dithizone and DMSO were obtained from Sigma.

90% ethanol was prepared by mixing ethanol and water 90:10 v/v. MTT was dissolved (1 mg/ml) in PBS. Dithizone was dissolved (0.0025 g/ml) in DMSO to form a stock solution. Dithizone working solution was prepared by adding 3 μl/ml dithizone stock to PBS.

The reagent filled tray is sealed with a plastic laminated, tear-off metal foil lid that is welded onto the tray (165-175° C.; 2.4 seconds). The assay components were then sterilised by gamma irradiation.

TABLE 5
Reagents used in the liver biopsy functional viability assay
that compares the metabolic activity of test liver biopsies
with devitalised negative control biopsies.

| | Column 1. | Column 2. | Incubation |
|---|---|---|---|
| Row | Test medium | Negative control | time (min) |
| 1 | PBS | 90% ethanol | 5 |
| 2 | Dithizone working | Dithizone working | 6 |
| | solution | solution | |
| 3 | MTT | MTT | 8 |
| 4 | NBF | NBF | 5 |
| 5 | PBS | PBS | 5 |

Viable liver biopsies stain purple whereas non-viable biopsies are light brown. The output of the assay may be assessed visually to obtain a rapid qualitative result, assessed semi-quantitatively by comparison to a ‘colour chart’ or quantified by image processing.

Devitalised liver biopsies (killed with 90% ethanol) serve as a negative control during visual inspection and assist in calibration during image processing.

The assay cassette allows replicate test and control samples to be assessed, thereby minimising the chance the result will be compromised by sampling errors. It also allows different regions of an organ to be biopsied and assessed simultaneously.

### Example 18

It is possible to use alternative combinations of histochemical dyes or biological reagents to assess the function of liver cells and liver tissue biopsies.

A combination of eosin (0.05%; 5 min) and MTT (1 mg/ml; 30 min) was used to double-label live and dead HUH-7 hepatocyte-like cells in PBS plus 0 mM glucose. (Note: results showed that liver samples and hepatocyte-like cells retained the ability to metabolise MTT in the absence of exogenous nutrients for the duration of the assay. This might be because in the absence of nutrients liver cell metabolism is stimulated by a need to increase gluconeogenesis).

Eosin (pink) is a charged molecule and so is excluded from viable cells with intact membranes. In contrast it is able to enter and stain non-viable cells with permeable membranes. Conversely MTT stains viable cells blue and leaves non-viable cells unstained. Combinations of the two dyes therefore stain dead liver red and viable liver blue.

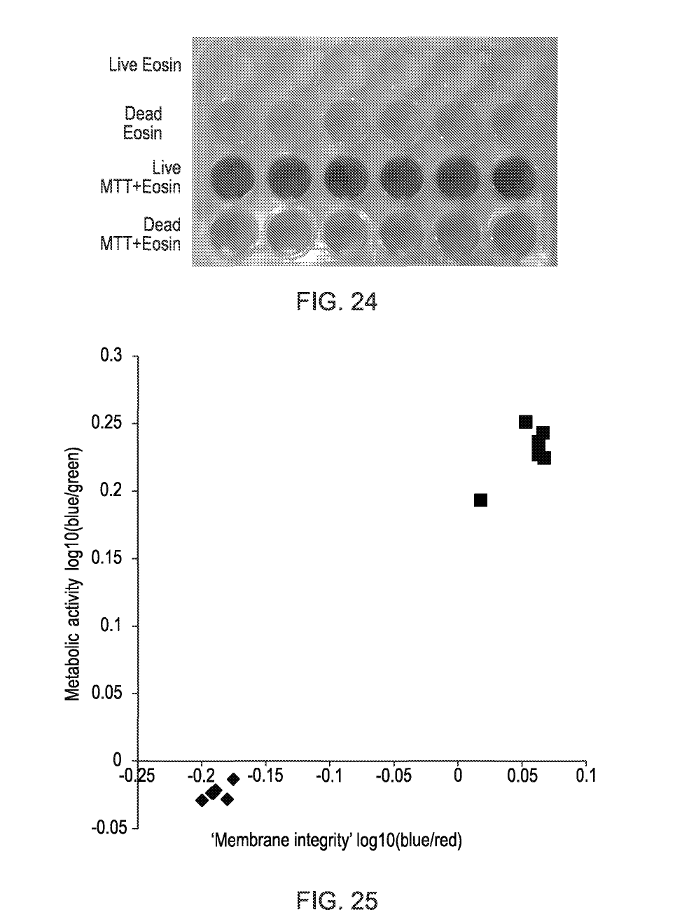

FIG. 24 shows live and devitalised HUH-7 hepatocyte-like cells stained with eosin and MTT.

For image processing, MTT staining may be quantified using the parameter log 10(blue/green) whereas Eosin staining may be quantified as changes in log(blue/red).

FIG. 25 illustrates how a combination of these two calculations can discriminate between viable and devitalised liver cells.

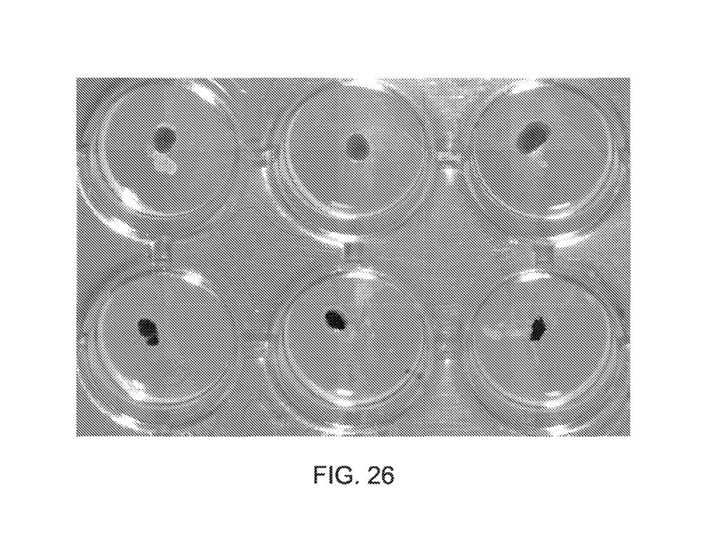

FIG. 26 shows a monochrome representation of live and dead liver tissue stained with combinations of eosin and MTT. Results show that the live liver is stained bright blue whereas the dead liver is stained bright red.

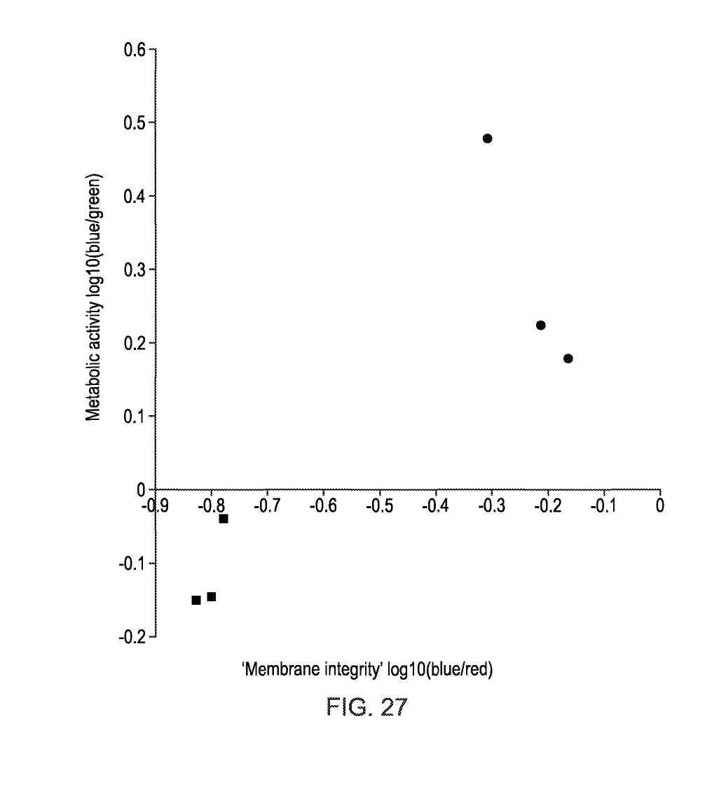

FIG. 27 shows that MTT staining may be quantified using the parameter log 10(blue/green) whereas Eosin staining may be quantified as changes in log(blue/red). This combination of calculations may be used to distinguish between live and dead liver samples.

### Example 19

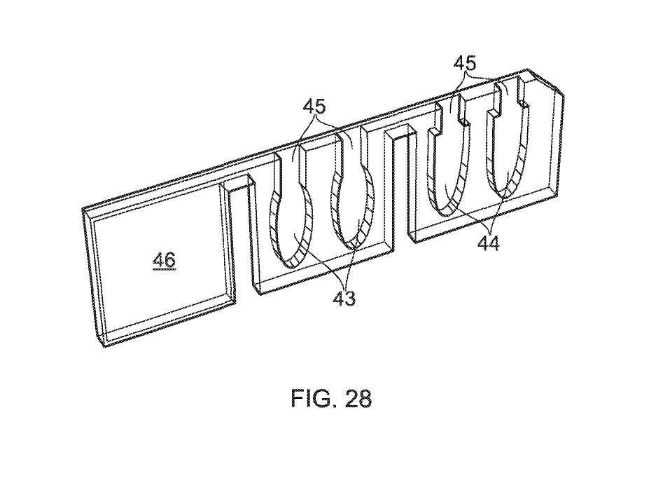

FIG. 28 shows an assay cassette for quality control assays of biopsies taken from solid organs, (e.g. liver, lung, kidney or heart) prior to transplantation. It allows the viability of two duplicate test biopsies (43) to be compared with two duplicate negative control biopsies (44).

The geometry of the inlet ports (45) of the assay cassette shown in FIG. 28 is designed to facilitate placing needle biopsies within the chambers. Furthermore, the geometry of the assay cassette shown in FIG. 28 (in contrast to the design shown in FIG. 11) allows it to be used with the reagent tray as shown in FIG. 2b and the calibration ‘square’ shown in FIG. 13. In this example the calibration square is positioned as shown in FIG. 28, item 46.

### Example 20

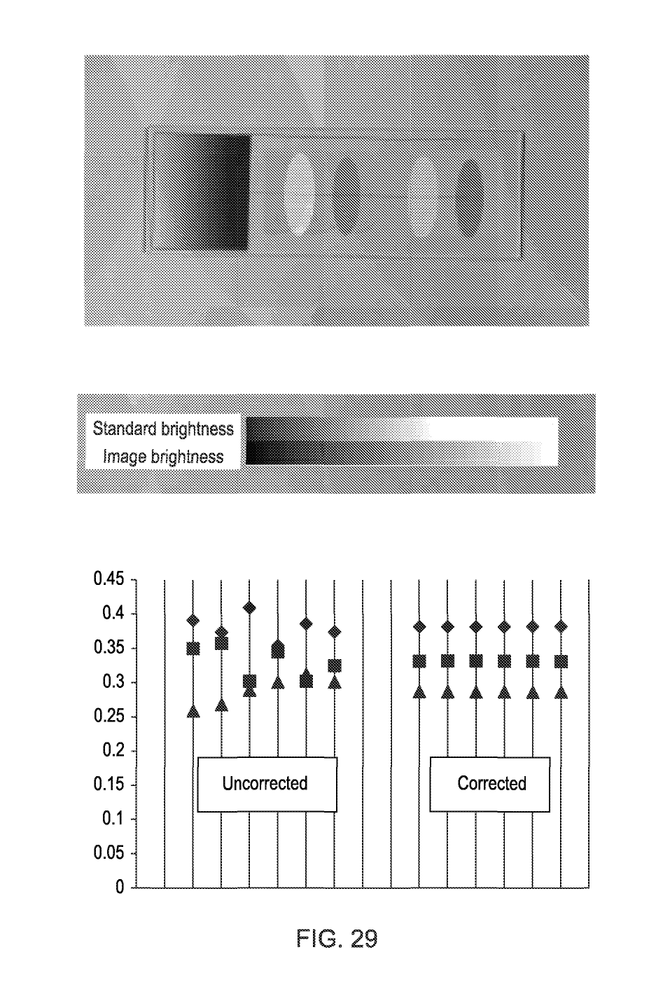

FIG. 29 shows the use of the ‘calibration square’ (as in FIG. 13) in the calibration of a digital photograph of the assay cassette. The top panel shows how recognition of the features in the calibration square (e.g. the red, green, blue and black lines surrounding the central graduated grey region) permits software to estimate the midline and linear dimensions of the assay cassette, and from this information, predict the locations of the sample chambers.

The middle panel of FIG. 29 shows how analysis of the central grey region of the calibration allows the intensities of pixels in a digital image to be related to the ‘true’ intensities of the points in the calibration standard.

The bottom panel of FIG. 29 shows how the calibration square can be used to correct the colour balance of photographs taken under different lighting conditions. A standard ‘blue’ material square was photographed under different lighting conditions ranging from tungsten bulbs (i.e. light with a strong ‘red-cast’) to a laboratory illuminator (set to produce light with an unusually strong blue-cast) using a Panasonic digital camera. The six ‘uncorrected’ data sets on the left side of the figure show the relative red (square), green (triangle) and blue (diamond) pixel intensities for the six photographs of the standard blue square. The six ‘corrected’ data sets on the right side of the figures are calculated by the software based upon the assumption that the central portion of the calibration square is grey and that therefore any inequality in the red, green and blue pixel intensities in the photograph must be an artefact caused by colour in-balance in the background illumination.

### Example 21

Example 21 demonstrates that L929 cell aggregates can be used as reproducible, surrogate test specimens for establishing islet viability assays to show (i) measurement of viable aggregate number, (ii) measurement of the loss of viability induced by adverse culture conditions (e.g. transient anoxia or nutrient deprivation), and (iii) use of alternative viability dyes in the assay of the invention.

Clinical samples of pancreatic islets are only infrequently available for laboratory studies and, when available, are of highly variable quality. L929 aggregates were therefore used as surrogate ‘test specimen’ to establish a reproducible baseline for the islet assay.

Poly(HEMA) solution (1.0 g in 50 ml of ethanol; 37° C.; Sigma P3932) was aliquoted (100 μl) to the wells of a 12 well plate and air dried to create a highly non-cell adherent surface.

A confluent T75 flask of L929 cells was trypsinised, the released cells diluted to 12 ml with DMEM/10% FCS and the resulting cell suspension aliquoted (1 ml) to the wells of the poly(HEMA) coated 12 well plate. DMEM/10% FCS (4 ml) was added to each well and the cells allowed to form into aggregates for 2-4 days in a humidified atmosphere of 5% CO₂ at 37° C.

Microscopic examination indicated that the L929 cells formed aggregates that were of a similar size range to human islet preparations (i.e. 70 μm-180 μm; mean 150 μm). Suspensions of the L929 aggregates were aliquoted into 70 μm cell strainers (0-8 ml aliquots) or into the wells of the assay cassettes (150 μl) and stained to determine their viability.

For staining method 1, L929 aggregates were stained by sequential immersion in (i) a wash solution (PBS; 5 min); (ii) a high nutrient medium (PBS containing 25 mM glucose and non-essential amino acids; 40 min); (iii) a metabolic indicator (MTT, 2 mg/ml; 15 min); (iv) a fixative (e.g. neutral buffered formalin; 5 min); and (v) a second wash solution (e.g. PBS; 5 min). The stained aggregates were photographed using a digital camera and the images analysed by image processing.

For staining method 2, L929 aggregates were stained with Calcein AM (a green fluorescent marker of live cells; Invitrogen, 2 μl/ml) and Ethidium bromide HD (a red fluorescent marker of dead cells; Invitrogen, 0.5 μl/ml) for 60 minutes. The samples were then rinsed in PBS and viewed using a confocal microscope.

To produce samples of L929 aggregates with different levels of viability, L929 aggregates were cultured in either (i) standard medium in a humidified atmosphere of 5% CO₂ at 37° C. (viable control); (ii) exposed to 70% ethanol in PBS for 5 min (i.e. the devitalising solution used to produce the assay's negative control); (iii) exposed to transient anoxia (a major cause of loss of transplant viability; 1% oxygen in a humidified atmosphere of nitrogen containing 5% CO₂ for 16 hours followed by a return to normal levels of atmospheric oxygen); or (iv) starved of nutrients for 4 weeks.

FIG. 31 shows the different levels of viability in L929 aggregates cultured under conditions (i) to (iv).

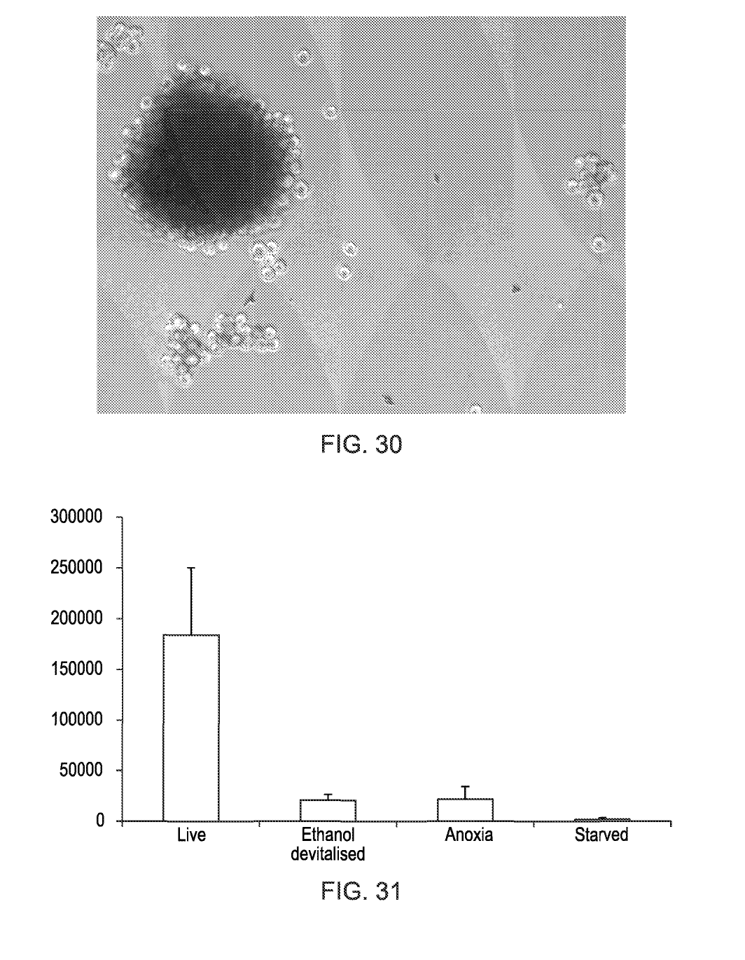

FIG. 30 shows that staining method 1 gave pronounced dark blue staining of the viable L929 aggregates. Image analysis (and visual observation) showed that the amount of staining was proportional to the number of viable aggregates present in the sample. The devitalising solution used in the assay abolished the blue staining to leave a yellow/brown colour in the negative controls. L929 aggregates that had been exposed to transient anoxia showed markedly reduced levels of blue staining. Nutrient starved samples showed an absence of blue staining.

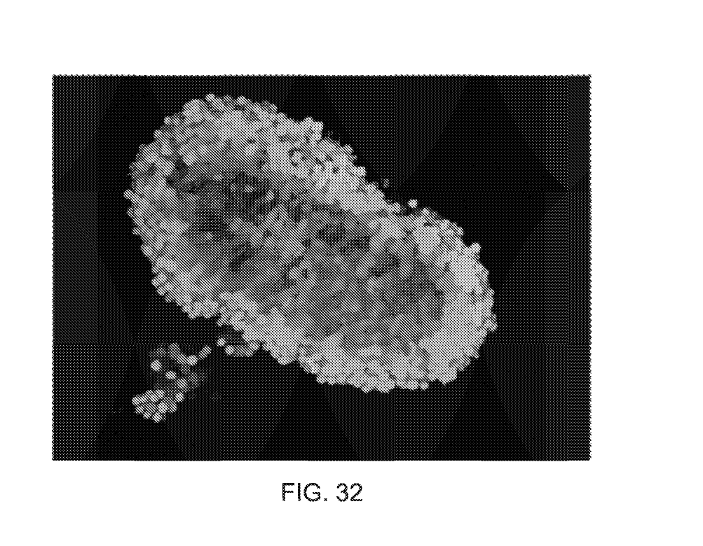

Likewise, FIG. 32 shows that viable L929 aggregates were stained fluorescent green (but not red) by the combination of calcein AM and ethidium bromide HD using staining method 2. Dead cell nuclei stained red.

Comparison of staining method 1 and staining method 2 showed that both dye combinations could be used to determine the viability of an L929 aggregate sample using the method of the invention. The data therefore shows that the method of the invention can use different chromogenic reagents to measure viability in the same test material.

For practical purposes the visible spectrum dyes were preferred to the fluorescent dyes because there was no need to use a fluorescent microscope to analyse the results.

### Example 22

The assay of the invention was also used to assess tissue viability of heart, kidney and lung tissues.

Rats (300-350 g) were humanely euthanized by cervical dislocation and their hearts, kidneys and lungs removed. Because it is difficult to take reproducible needle biopsies of rodent organs using standard clinical equipment, each organ was finely minced to simulate needle biopsies. Portions of minced organ were rinsed in PBS and either (i) immersed in ethanol (70%, 5 min; devitalised controls); (ii) immersed in PBS (5 min; viable specimens); or (iii) damaged by freeze thawing three times (cycling between −80° C. and room temperature for >30 min).

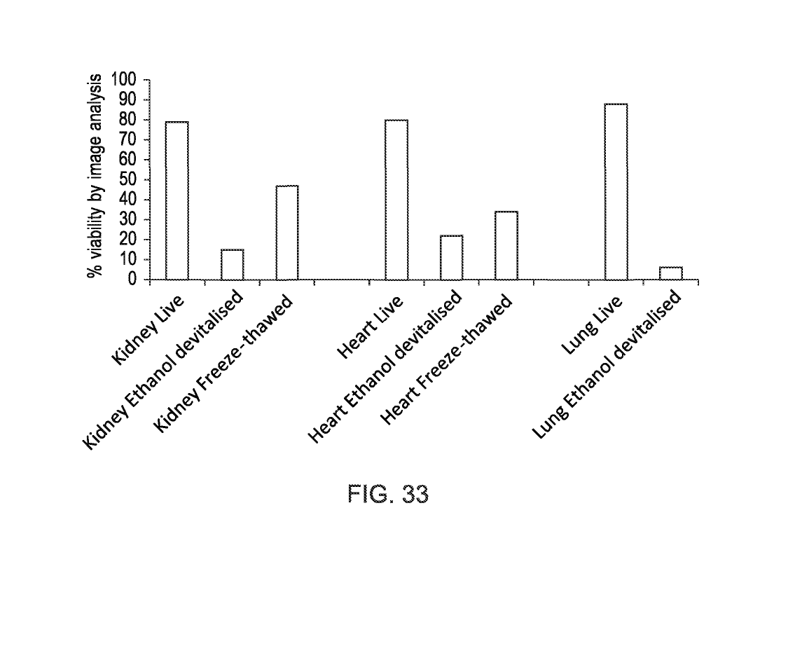

The treated organ fragments were then stained using a solution of MTT (2 mg/ml) and dithizone (7.5 micrograms/ml) in PBS for various time periods, rinsed in PBS (1 min) and fixed in neutral buffered formalin (10 min). Fixed and stained samples were then rinsed in PBS and photographed. The results are shown in FIG. 33.

Results showed that MTT plus dithizone stained viable kidney fragments an intense dark purple/blue. Staining became apparent after 30 seconds and was maximal after 5 minutes with optimal staining being observed between 90 seconds and 2 minutes. By comparison the ethanol killed samples showed greatly reduced levels of staining. Image analysis revealed that this difference in staining could be easily quantified (e.g. by comparing the red pixel intensity values) and was highly statistically significant. The samples that had been damaged by freeze-thawing showed greatly reduced levels of staining that were close to, (but statistically distinguishable from), the ethanol-devitalised controls.

Heart fragments stained more slowly and less intensely than kidney, with initial staining observed after 1 minute, maximal staining after 5 minutes, and optimal staining observed after 2 minutes. Viable heart samples stained an intense dark blue. By comparison, the ethanol devitalised heart samples and heart samples that had been damaged by freeze-thawing were less intensely stained.

Image analysis showed that the differences in staining intensities between viable and non-viable heart samples could be quantified and that these differences were statistically significant. Viable heart samples stained blue-ish purple, whereas dead heart samples stained red-ish purple, allowing the colour (i.e. hue) of the samples to be used as an additional measure of viability.

Lung fragments stained most slowly, with initial staining only becoming apparent after 3 minutes, maximal staining after 10 minutes and optimal staining being observed after 5 minutes. Ethanol devitalised samples showed statistically significantly reduced staining compared with viable samples.

Overall, these results show that it is possible to use a generic staining protocol to test the viability of solid organ samples (e.g. lung, heart and kidney). When performed with the apparatus of the invention, this creates a ‘general purpose’ assay for the viability of needle biopsies collected from solid organs (e.g. from organs intended for use in transplant procedures).

### Example 23

The viability assays of the invention use a chromogenic set of reagents to produce a readily measurable colour change that reflects the viability of a tissue sample prior to its use in a therapeutic procedure. The reagent design of the assay was optimised to identify permissible ranges and functional requirements for each reagent as follows.

The reagents used in a typical pancreatic islet viability assay in accordance with the invention are listed in table 6. However, the principles discussed below also apply to other cell types.

TABLE 6
reagents typically used in pancreatic islet viability assay

| | Test sample | Negative control |
|---|---|---|
| Reagent 1 | PBS | 70% ethanol in PBS |
| Reagent 2 | PBS containing 25 mM | PBS containing 25 mM glucose and |
| | glucose and non-essential | non-essential amino acids |
| | amino acids | |
| Reagent 3 | MTT (2 mg/ml) | MTT (2 mg/ml) |
| Reagent 4 | Neutral buffered formalin | Neutral buffered formalin |
| Reagent 5 | PBS | PBS |

Reagent 1 (Test Sample): A Wash Reagent that Removes the (Undefined) Transport Medium that Previously Held the Cell Sample e.g. Islet Sample.

The isolated islets arrive in a transport medium (e.g. University of Wisconsin Solution), but the composition of that solution will be unknown and cannot be specified in advance. Furthermore, the nutrients in the transport medium will have been consumed to an unpredictable extent whilst adverse materials may have accumulated to potentially toxic levels (e.g. inflammatory cytokines, cell debris and metabolic waste products). The transport medium must therefore be removed using a ‘wash solution’ before the assay begins. The wash solution is typically sterile, chemically stable (i.e. have a shelf-life of >6 months at 4° C.), isotonic and biocompatible (i.e. composed of cell culture grade non-cytotoxic materials). Preferably it should be buffered to maintain pH between 6.4 and 7.8 at room temperature in a normal air atmosphere (i.e. without the need for a humidified atmosphere containing 5% CO₂). It should not contain any of the strongly coloured components that are sometimes added to culture media (e.g. phenol red).

The test sample is preferably incubated with PBS (or equivalent wash reagent) for between 1 minute and 15 minutes, preferably 3 to 7 minutes, more preferably approximately 5 minutes.

Results show that Dulbecco's phosphate buffered saline (Sigma D8537) should be selected as the preferred wash solution (R1). However, it is expected that other neutral phosphate buffered saline solutions could be used instead without adversely influencing the results.

Reagent 1 (Negative Control): A Devitalising Wash Reagent that Both Removes the Transport Medium and Devitalises the Cell Sample e.g. an Islet Sample to Produce a ‘Negative Control’ Sample Against which a Test Sample of Unknown Viability May be Compared.

The devitalising reagent should rapidly kill the cells in the negative control sample (i.e. permanently reduce their metabolic activity to basal levels). It should not dramatically alter cell morphology (e.g. detergents and other cell lysis solutions would be inappropriate). Preferably, the devitalising reagent is not so toxic that it creates a hazard to health during the assay's use or subsequent waste disposal (e.g. azide may not be desirable).

Results show that 70% ethanol in the wash solution (e.g. 70% ethanol in Dulbecco's phosphate buffered saline) is a preferred devitalising/wash solution. A one minute exposure to this solution is sufficient to reduce the negative control sample's metabolic activity to background levels. Preferably, the exposure is between 1 to 15 minutes, more preferably 3 to 7 minutes, most preferably approximately 5 minutes. The 5 minute incubation period typically used herein therefore provides a ‘safety margin’ to mitigate against user variability, impatience or error. Longer time periods (e.g. 7 minutes or 15 minutes) are functional and do not have any adverse consequences beyond introducing unnecessary delay.

A range of ethanol concentrations can be used with the negative control, provided that the cells in the sample are devitalised e.g. 50-100%, preferably 70-100%, most preferably 70-95%.

Reagent 2: A High Nutrient Reagent that Induces Maximal Metabolic Activity within Viable Islets.

After post-mortem recovery of cells e.g. pancreatic cells from an organ donor, enzymatic isolation of the islets from the donated pancreas and (potentially) prolonged exposure to sub-optimal cell culture conditions (e.g. transport between hospitals in University of Wisconsin Solution) an islet sample is likely to be traumatised and temporarily metabolically dormant, irrespective of its actual viability. (For this reason, assays that measure a clinical islet sample's metabolic activity at the point of use without first correcting for this issue are likely to return a low value or even a false negative result).

Reagent 2 in the assay therefore incubates the islets in high nutrient media to allow the islet sample to recover and achieve its maximum level of metabolic activity.

Because islets are essentially nutrient sensors that respond to excessive levels of circulating nutrients with a metabolically intense burst of insulin secretion, maximal metabolic activity can be achieved by exposing the islets to high levels of nutrients, especially combinations of glucose and amino acids.

Results from studies using human, sheep and rat islets indicate that exposure to high nutrient PBS (i.e. PBS supplemented with 25 mM glucose plus non-essential amino acids) for approximately 40 minutes constitutes a simple, chemically-defined mechanism for inducing high metabolic activity in previously quiescent isolated islets. This solution will also maintain the islets' pH without the need for a CO₂ buffered incubator.

The concentration range of the non-essential amino acids in the high nutrient reagent may vary. Preferably, the non-essential amino acids are used at manufacturers recommended concentration as a 1 in 100 v/v dilution of commercially available stock (i.e. 1%). However, a 0.5% to 5% solution may also be used.

The concentration range for glucose in the high nutrient reagent may also vary. It is desirable to maximally stimulate insulin production (via increased Beta islet cell metabolism) so a concentration range of 17 mM-25 mM glucose is desirable (although higher concentrations may also be used (e.g. up to 50 mM).

Additional components may be added to this nutrient reagent to provide a more complete nutrient mixture with a broader range of stimuli, (e.g. by adding 10% Ham's F-12).

Results also showed, surprisingly, that although islets respond to changes in nutrient levels very rapidly in vivo, (i.e. <=15 minutes) much longer time periods of exposure to high nutrients are required to restore full metabolic activity to traumatised/quiescent isolated islet samples in vitro. Preferred time periods for incubation in reagent 2 are therefore >30 minutes, ideally 40-45 minutes. Longer time periods (60-90 minutes) are likely to have beneficial effects in scientific studies because they will provide a more stable baseline, but are likely to introduce unnecessary delays when the assays are used in a clinical situation.

It is possible to add additional histological stains to reagent 2 in order to improve the sensitivity and discriminating power of the assay (e.g. dithizone). When islets produce insulin it is stored as a zinc complex prior to its release. The pink coloured zinc chelating reagent dithizone therefore stains insulin-rich islets pink. In contrast, since dead/dying islets degranulate and release their insulin in an uncontrolled manner, dysfunctional islets show much lower levels of dithizone staining.

Dithizone staining is a comparatively slow process and therefore it would be undesirable to include it as a separate step at this would delay the results from the assay. It can however be added to reagent 2 to give an additional marker of islet functional viability without creating additional delay.

Reagent 3: A Cell Viability Indicator Reagent (e.g. MTT).

Results showed that a 15 minute incubation in MTT (2 mg/ml in PBS) produces a reliable colour change in viable islets. Viable, metabolically active islets are stained blue by this reagent. By comparison, the devitalised islets in the negative control sample are unstained or stained faintly brown-yellow. Because islets stained with MTT are visible to the naked eye (as small dark dots) the use of this dye allows a clinician to gain an initial, qualitative result before the assay is complete and quantification has provided a detailed numerical output.

The use of an insoluble metabolic dye also allows the number and metabolic activity of individual islets within a sample to be assessed. This is an improvement over alternative laboratory techniques (e.g. oxygen consumption rate per unit DNA) which can only give a measure of the islet sample's overall properties.

Results showed that MTT concentrations of 1-2 mg/ml and time periods of 10-20 minutes could be used in the assay. It was observed that there was an initial ‘lag’ period of ˜5 minutes before MTT staining became visible and it was therefore concluded that incubation periods of 10 minutes or less would be vulnerable to increased variability if the incubation time was incorrectly applied. By contrast, the staining produced by an incubation time of 15 minutes would less vulnerable to small errors by the assay's users.

Likewise, results showed that whilst 1-2 mg/ml MTT gave suitable results, a 2 mg/ml solution provided a marked excess of reagent and was therefore less likely to be adversely affected by small losses of reagents during assay storage.

Reagent 4: A Fixative Reagent to Terminate the Staining Reaction and Kill any Pathogens Present in the Cell Sample e.g. Islet Sample.

Results showed that a 15 second to 15 minute exposure to neutral buffered formalin terminated the staining reaction. A 5 minute incubation period was therefore selected to ensure that user impatience did not result in inadequate fixation. Results showed that prolonged fixation times (5 to 15 minutes) did not leach the coloured reaction products from the sample. Typically, 10% NBF is used for 5 minutes (as supplied by the manufacturer. This is 3.7%-4.0% formaldehyde w/v in PBS). However, a range of concentrations may be used, from e.g. 1%-10%. Furthermore, a range of fixation times may also be suitable e.g. from 4 minutes to 30 minutes. (Note formaldehyde is a gas that dissolves in water to give a 37% solution w/v, so a 1 in 10 dilution of this solution gives 3.7%-4.0% formaldehyde w/v).

Reagent 5 an Additional Wash Reagent to Remove the Spent Reagents Prior to the Assessment of the Results.

Results showed that there was little or no difference between washing the assay cassette in water or PBS at the end of the assay. It was decided to use PBS for the washing step in order to streamline manufacture and quality management by reducing the number of reagents. The use of PBS will also remove the risk of fluctuation in pH associated with the use of distilled or deionised water.

A range of incubation times may be used e.g. 1 to 45 minutes, preferably 1 to 5 minutes.

### Example 24

The assay kit may include (i) an assay cassette as described herein; (ii) a reagent tray as described herein and optionally (iii) a reference standard e.g. a printed ‘calibration strip’ that is attached to a specific location on the assay cassette (e.g. the handling portion). To determine the functional viability of a tissue intended for use in a therapeutic procedure, samples of the tissue are placed into the chambers of the assay cassette and sequentially immersed into a series of reagents contained within the reagent tray in order to produce a colour change in the tissue samples that is proportional to the samples' functional viability.

At the end of the procedure, the intensity of the colour change is quantified by image analysis of a single digital photograph of the assay cassette that shows both the calibration strip and the stained samples contained within the assay cassette.

The image analysis is a four stage process comprising (i) identification of the major features of the image (e.g. the calibration grid and sample chambers); (ii) calibration of the image in terms of pixel intensity, colour balance and spatial geometry using the calibration grid as a reference; (iii) identification and measurement of the tissue samples within the reaction chambers; and (iv) summation and presentation of the results as both an easily comprehensible graphic (for immediate use within a clinical environment) and as a detailed spreadsheet (for subsequent study and analysis).

The first stage of the image analysis program identifies and locates the calibration strip within the overall image and uses the calibration strip to gain spatial information (e.g. number of pixels per mm), intensity information (e.g. pixel intensities in the image corresponding to white and black) and colour information (e.g. colour cast caused by tungsten lighting). The ‘calibration strip’ may be recognised within the overall image by locating specific and distinctive patterns of grey-scale intensity and colour saturation on the calibration strip (e.g. by identifying the black, red, blue and green lines at the periphery of the calibration strip). This stage of the process may begin with a ‘low-resolution’ scan of the overall image to determine approximate, ‘candidate’ positions for the calibration strip followed by more detailed analysis of small ‘regions of interest’ within the image in order to identify and verify the precise location of the calibration strip. This stage of the process may include error trapping routines to reject markedly flawed images (e.g. assay cassette photographed upside down).

In the second stage, the image may be corrected (e.g. for white balance and uneven illumination). A ‘look up table’ is then created to relate the intensity of pixels in the image to the ‘known’ properties of the calibration strip (e.g. the observed red, green and blue intensities of pixels at the left hand side of the calibration grid correspond to white ‘in the real world’ whilst the observed red, green and blue intensities of pixels at the right hand side correspond to black ‘in the real world’). Analysis of the calibration grid also gives spatial information about the image (e.g. the number of pixels between the red line and the black line in the calibration strip is the number of pixels equivalent to 20 mm). Likewise, the average slope and position of the blue line and the green line in the calibration grid give the mid-line of the assay cassette. (It is possible to include additional error checking steps at this stage to ensure that the program is functioning correctly).

The third stage locates the tissue samples and measures their staining intensity using the calibration strip as a reference standard. Since the ‘real world’ geometry of the assay cassette, the ‘real world’ location of the calibration strip on the assay cassette, the location of the calibration strip in the image and the number of pixels per mm are now known, it is possible to calculate the position of a point within each of the sample chambers within the digital image of the assay cassette and estimate the approximate position of the edges of the wells. Additional image processing steps then confirm the exact margins of the sample wells. (Note it is possible to place coloured marks on the reagent tray to assist in this stage of the process).

The areas of the image within the sample wells are then analysed. Pixels within these areas are classified as either foreground (i.e. part of the tissue) or background (i.e. not part of the tissue), for instance, by using local threshold-based methods. In the case of tissue samples that consist of numerous small aggregates of cells (e.g. pancreatic islets) it is necessary to use ‘blob detection’ and ‘equivalence’ algorithms to identify each foreground object as a distinct foreground entity.

Numerical data are then collected on the foreground entities (e.g. number of objects, the height, width, area and staining intensity of each object and the total staining intensity for the whole sample). In situations where the tissue sample is compared to a devitalised negative control, these procedures are also used to assess the negative control specimen.

In the final stage, the numerical data is summarised into (i) a format that can be readily understood by clinicians in a stressful clinical environment; and (ii) a format appropriate for scientists engaged in research. For instance, in the case of pancreatic islets, the approximate size and staining properties of ‘healthy’ and ‘dead’ human islets are already known. It is therefore possible to present the data from the assay to a clinician as a readily comprehensible ‘traffic light’ graph. This displays the data from each individual islet in a sample as a point in the green region of the graph if the islet appears to be healthy, in the red region of the graph if its size or staining properties suggest that it is dead or in the orange region if it has intermediate properties. The number and distribution of points on the ‘traffic light’ graph therefore gives clinicians a rapidly comprehensible summary of the heath of an islet sample.

The precise numerical data can also be exported to an Excel spreadsheet for subsequent detailed analysis.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1-68. (canceled)

69. An assay kit comprising:
(i) an assay cassette comprising at least one reaction chamber housing that encloses at least one reaction chamber, the reaction chamber having an access port for introducing the biological sample therein and a permeable barrier that permits fluid exchange into and out of the reaction chamber whilst retaining the cells of the biological sample within the reaction chamber, the assay cassette further comprising a handling portion;
(ii) a reagent vessel comprising a multiwell plate comprising an array of reagent chambers for holding reagents, each reagent chamber comprising a pair of side walls, front and rear walls, and a bottom wall;
(iii) a reference standard attached to the assay cassette; and
(iv) instructions for use in assessing the viability of a biological sample.

70. The kit according to claim 69, wherein the reagent vessel comprises:
a) at least one reagent chamber having a wash reagent therein and
b) at least one reagent chamber having a cell viability indicator reagent therein.

71. The kit according to claim 69, wherein the reagent vessel comprises:
a) at least one reagent chamber having wash reagent therein,
b) at least one reagent chamber having a cell viability indicator reagent therein, and
c) at least one reagent chamber having a fixative reagent therein.

72. The kit according to claim 69, wherein the reagent vessel comprises:
a) at least one reagent chamber having wash reagent therein,
b) at least one reagent chamber having a nutrient reagent therein
c) at least one reagent chamber having a cell viability indicator reagent therein, and
d) at least one reagent chamber having a fixative reagent therein.

73. The kit according to claim 69, wherein the reagent vessel comprises:
a) at least one reagent chamber having wash reagent therein,
b) at least one reagent chamber having a nutrient reagent therein
c) at least one reagent chamber having a cell viability indicator reagent therein,
d) at least one reagent chamber having a fixative reagent therein, and
e) at least one reagent chamber having a further wash reagent therein.

74. The kit according to claim 69, wherein the reference standard is for calibrating the visualization of a first colorimetric signal and/or a second colorimetric signal in terms of brightness, orientation, linear dimensions and/or colour balance.

75. The kit according to claim 69, wherein the reference standard is attached to the assay cassette such that in use it may be positioned adjacent the reaction chambers.

76. The kit according to claim 75, wherein the reference standard is attached to the handling portion of the assay cassette.

77. The kit according claim 69, wherein the reference standard is a multi-coloured strip.

78. An apparatus for assessing viability of a biological sample comprising cells, the apparatus comprising:
(i) a reagent vessel comprising mutually independent interconnected rows and columns of reagent chambers for containing reagents, each reagent chamber being defined by a front and a rear wall, a pair of side walls and a bottom wall, the reagent vessel comprising a partitioning wall being defined by a side wall of adjacent columns of reagent chambers, the reagent vessel further comprising a means for ensuring correct orientation of an assay cassette within the reagent chambers, wherein the reagent vessel comprises at least one reagent chamber having a cell viability indicator reagent therein, wherein the at least one reagent chamber having a cell viability indicator reagent therein has a removable cover element; and
(ii) an assay cassette comprising at least one reaction chamber housing that encloses at least one reaction chamber, the reaction chamber having an access port for introducing the biological sample therein and a permeable barrier that permits fluid exchange into and out of the reaction chamber whilst retaining the cells of the biological sample within the reaction chamber, the assay cassette further comprising a reference that in use it may be positioned adjacent the reaction chambers.

79. The apparatus according to claim 78, wherein the reagent vessel comprises:
a) at least one reagent chamber having a wash reagent therein and
b) at least one reagent chamber having a cell viability indicator reagent therein.

80. The apparatus according to claim 78, wherein the reagent vessel comprises:
a) at least one reagent chamber having wash reagent therein,
b) at least one reagent chamber having a cell viability indicator reagent therein, and
c) at least one reagent chamber having a fixative reagent therein.

81. The apparatus according to claim 78, wherein the reagent vessel comprises:
a) at least one reagent chamber having wash reagent therein,
b) at least one reagent chamber having a nutrient reagent therein
c) at least one reagent chamber having a cell viability indicator reagent therein, and
d) at least one reagent chamber having a fixative reagent therein.

82. The apparatus according to claim 78, wherein the reagent vessel comprises:
a) at least one reagent chamber having wash reagent therein,
b) at least one reagent chamber having a nutrient reagent therein
c) at least one reagent chamber having a cell viability indicator reagent therein,
d) at least one reagent chamber having a fixative reagent therein, and
e) at least one reagent chamber having a further wash reagent therein.

83. The apparatus according to anyone of claim 78, wherein the reference standard is attached to the handling portion of the assay cassette.
